(19) United States
(12) Patent Application Publication
(10) Pub. No.: US 2010/0004693 A1
(43) Pub. Date: Jan. 7, 2010

(54) CAM LOCKING SPINE STABILIZATION SYSTEM AND METHOD

(76) Inventors: Peter Thomas Miller, Austin, TX (US); Jeremy J. Lemoine, Austin, TX (US)

Correspondence Address: SPRINKLE IP LAW GROUP, 1301 W. 25TH STREET, SUITE 408, AUSTIN, TX, 78705, US

(21) Appl. No.: 12/165,724

(22) Filed: Jul. 1, 2008

(51) Int. Cl. A61B 17/04; A61B 17/70

(52) U.S. Cl. 606/308; 606/301; 606/305; 606/265

(57) ABSTRACT

A spine stabilization may include a rod and bone fastener assemblies. Each bone fastener assembly may include a bone fastener and a collar. Each bone fastener may have a threaded shank and a head. Each collar may have a first end with a cavity for accommodating the bone fastener and a second end having a channel for accommodating the rod. The channel may have a first portion for positioning the rod. The channel may have a second portion for advancing the rod, wherein rotating the collar advances the rod in the channel.

### TECHNICAL FIELD OF THE DISCLOSURE

The present disclosure generally relates to spinal stabilization systems, and in particular to spine stabilization systems in which a rod may be positioned in a collar and the collar may be rotated to secure the rod in the collar. Embodiments of the disclosure may cause cold welding between components of the spine stabilization system to inhibit movement between components.

### BACKGROUND OF THE DISCLOSURE

Bone may be subject to degeneration caused by trauma, disease, and/or aging. Degeneration may destabilize bone and affect surrounding structures. For example, destabilization of a spine may result in alteration of a natural spacing between adjacent vertebrae. Alteration of a natural spacing between adjacent vertebrae may subject nerves that pass between vertebral bodies to pressure. Pressure applied to the nerves may cause pain and/or nerve damage. Maintaining the natural spacing between vertebrae may reduce pressure applied to nerves that pass between vertebral bodies. A spinal stabilization procedure may be used to maintain the natural spacing between vertebrae and promote spinal stability.

Spinal stabilization may involve accessing a portion of the spine through soft tissue. Conventional stabilization systems may require a large incision and/or multiple incisions in the soft tissue to provide access to a portion of the spine to be stabilized. Conventional procedures may result in trauma to the soft tissue, for example, due to muscle stripping.

Spinal stabilization systems for a lumbar region of the spine may be inserted during a spinal stabilization procedure using a posterior spinal approach. Conventional systems and methods for posterolateral spinal fusion may involve dissecting and retracting soft tissue proximate the surgical site. Dissection and retraction of soft tissue may cause trauma to the soft tissue, and extend recovery time. Minimally invasive procedures and systems may reduce recovery time as well as trauma to the soft tissue surrounding a stabilization site.

### SUMMARY OF THE DISCLOSURE

A spinal stabilization system may be installed in a patient to stabilize a portion of a spine. A spinal stabilization system may be installed using a minimally invasive procedure. An instrumentation kit may provide instruments and spinal stabilization system components necessary for forming a spinal stabilization system in a patient.

Various instruments may be used in a minimally invasive procedure to form a spinal stabilization system in a patient. The instruments may include, but are not limited to, positioning needles, guide wires, dilators, bone awls, bone taps, sleeves, drivers, tissue wedges, rod length estimating tools, mallets, tissue retractors, and tissue dilators. The instruments may be provided in an instrumentation set. The instrumentation set may also include components of the spinal stabilization system. The components of the spinal stabilization system may include, but are not limited to, bone fastener assemblies of various sizes and/or lengths and rods.

A spinal stabilization system may be used to achieve rigid pedicle fixation while minimizing the amount of damage to surrounding tissue. In some embodiments, a spinal stabilization system may be used to provide stability to two or more vertebrae. A spinal stabilization system may include a rod and two or more bone fastener assemblies. Eache bone fastener assembly may include, but is not limited to, a bone fastener and a collar. A first portion of the bone fastener may couple to a portion of the spine during use. A first portion of a collar may couple to a second portion of the bone fastener. A second portion of the collar may couple to the rod during use. In some embodiments, an orientation of the bone fastener may be independent of the orientation of the collar for a bone fastener assembly. After the bone fastener is placed in a vertebral body, the collar coupled to the bone fastener may be positioned so that the rod can be positioned in the collar and in at least one other collar that is coupled to another vertebral body by a bone fastener. A rod may be positioned in a first portion of the channel. The collar may be rotated to advance the rod into a second portion of the channel. Rotation of the collar may cause cold welding between components of a spine stabilization system.

Some embodiments may enable cold welding to occur between components in a spine stabilization system. As used herein, the term “cold welding” may refer to joining two components in a spine stabilization system without heating the components. Cold welding may be described as a process in which joining takes place without fusion at the interface of two surfaces. In cold welding processes, pressure is applied to the components. In some embodiments, at least one of the mating parts is ductile. In some embodiments, the force of adhesion following first contact can be augmented by pressing the metals tightly together and/or increasing the duration of contact. In some embodiments, only the high points of each surface, called asperities, may touch the opposing piece. In some embodiments, as little as a few thousandths of a percent of the total surface is involved. However, these small areas develop powerful molecular connections and investigations of contact points reveal that an actual welding of the two surfaces takes place after which it is impossible to discern the former asperitic interface. If the original surfaces are sufficiently smooth the subatomic attractions between contact points eventually draw the two pieces completely together and may eliminate even the macroscopic interface.

Some embodiments provide a collar for coupling a spinal rod to a bone fastener. In some embodiments, the collar includes a first end and a second end. In some embodiments, the first end has an opening for accommodating a shank of a bone fastener and a cavity recessing from the opening into the first end for accommodating a head of the bone fastener. In some embodiments, the second end has a channel formed therein. In some embodiments, the channel has a first portion having a first interior geometric configuration to allow a rod to move relative to the collar. The first portion may be oriented in a plane at an angle relative to the longitudinal axis of the collar. The channel may also include a second portion oriented relative to the longitudinal axis of the collar. The second portion may have a second interior geometric configuration having a first side for biasing the rod against the head of a bone fastener and a second side for biasing the collar against the head of the bone fastener to cause cold welding of a surface of the cavity of the collar and a surface of the bone fastener and inhibit movement of the collar relative to the bone fastener. In some embodiments, the biasing of the first side of the collar and the surface of the rod further inhibits movement of the collar relative to the rod. In some embodiments, the first interior geometric configuration of the first portion of the channel comprises a width to provisionally lock the rod in the collar.

In some embodiments, the first portion of the channel is oriented parallel with the longitudinal axis of the collar. In some embodiments, the first portion of the channel is oriented perpendicular to the longitudinal axis of the collar. In some embodiments, the second geometric configuration of the second portion of the channel comprises a circular helix oriented about the longitudinal axis of the collar, such that the axial bias applied to the rod is proportional to a pitch and an arclength of the circular helix.

In some embodiments, the second portion of the channel is oriented perpendicular to the longitudinal axis of the collar, such that the axial bias applied to the rod is based on the width of the channel. In some embodiments, the second portion of the channel has a first width and the channel further comprises a recessed portion having a second width such that the second width is wider than the first width to inhibit removal of the rod. In some embodiments, the second portion of the channel has a first width and the channel further comprises a protuberance having a second width such that the second width is narrower than the first width to inhibit removal of the rod.

Some embodiments provide a method for coupling a rod to a bone fastener having a head and a shank. In some embodiments, the method may include advancing a bone fastener into a first end of a collar, positioning a rod in a first portion of a channel in a second end of the collar, advancing the rod into a second portion of the channel, and rotating the collar a selected angle about its longitudinal axis to bias the surface of the head of the bone fastener against the surface of the cavity. The first end of the collar may include an opening for accommodating a shank of the bone fastener and a cavity recessing from the opening into the first end for accommodating the head of the bone fastener. In some embodiments, the first portion has a first interior geometric configuration to allow the rod to move relative to the collar.

In some embodiments, the first portion is oriented in a plane at an angle relative to the longitudinal axis of the collar. In some embodiments, the second portion is oriented relative to the longitudinal axis of the collar and the second portion has a second interior geometric configuration having a first side for biasing the rod against the head of a bone fastener and a second side for biasing a surface of the cavity of the collar against the head of the bone fastener.

In some embodiments, rotating the collar a selected angle about its longitudinal axis to bias the surface of the head of the bone fastener against the surface of the cavity causes cold welding between a portion of the surface of the bone fastener and a portion of the surface of the cavity to inhibit movement of the collar relative to the bone fastener. In some embodiments, rotating the collar a selected angle about its longitudinal axis biases a surface of the rod against a surface of the channel, causing cold welding between a portion of the surface of the rod and a portion of the surface of the collar to inhibit movement of the collar relative to the rod. In some embodiments, advancing the rod in the first portion of the channel provisionally secures the rod in the collar. In some embodiments, the second geometric configuration comprises a circular helix oriented about the longitudinal axis of the collar, such that rotating the collar applies the axial bias to the rod proportional to a pitch and an arclength of the circular helix. In some embodiments, the second portion is oriented perpendicular to the longitudinal axis of the collar, such that rotating the collar applies an axial bias to the rod corresponding to the width of the channel. In some embodiments, the steps are part of a minimally invasive surgery (MIS) procedure.

Some embodiments provide a system for stabilizing a portion of a spine. In some embodiments, the system includes a rod having a length for spanning between two or more vertebrae, a plurality of bone fasteners and a collar. In some embodiments, each bone fastener comprises a head and a threaded shank for advancement into the two or more vertebrae. In some embodiments, a collar has a first end and a second end. In some embodiments, the first end has an opening for accommodating a shank of a bone fastener and a cavity recessing from the opening into the first end for accommodating a head of the bone fastener. In some embodiments, the second end has a channel formed therein. In some embodiments, the channel includes a first portion having a first interior geometric configuration to allow a rod to move relative to the collar.

In some embodiments, the first portion is oriented in a plane at an angle relative to the longitudinal axis of the collar. In some embodiments, the channel includes a second portion oriented relative to the longitudinal axis of the collar. In some embodiments, the second portion has a second interior geometric configuration having a first side for biasing the rod against the head of a bone fastener and a second side for biasing the collar against the head of the bone fastener to cause cold welding of a surface of the cavity of the collar and a surface of the bone fastener and inhibit movement of the collar relative to the bone fastener.

In some embodiments, the bone fastener has a head having an elliptical cross-section with a major axis and a minor axis. In some embodiments, the head of the bone fastener has a channel and a slot. In some embodiments, the channel is aligned with the minor axis and formed by two opposing arms along the major axis. In some embodiments, the cavity in the collar has an elliptical cross-section having a major axis and a minor axis, such that the width of the cavity on the major axis of the cavity is greater than the width of the head on the major axis of the head and the width of the cavity on the minor axis of the cavity is substantially equal to the width of the head on the major axis of the head. In some embodiments, a rod is securely coupled to the bone fastener and collar when the minor axis of the cavity is substantially aligned with the major axis of the head. In some embodiments, the biasing of the first side of the collar and the surface of the rod further inhibits movement of the collar relative to the rod. In some embodiments, the second geometric configuration of the second portion of the channel comprises a circular helix oriented about the longitudinal axis of the collar, such that the axial bias applied to the rod is proportional to a pitch and an arclength of the circular helix. In some embodiments, the second portion of the channel is oriented perpendicular to the longitudinal axis of the collar, such that the axial bias applied to the rod is based on the width of the channel.

Other objects and advantages of the embodiments disclosed herein will be better appreciated and understood when considered in conjunction with the following description and the accompanying drawings.

### BRIEF DESCRIPTION OF THE DRAWINGS

A more complete understanding of the present disclosure and the advantages thereof may be acquired by referring to the following description, taken in conjunction with the accompanying drawings in which like reference numbers indicate like features and wherein:

FIG. 1 depicts a perspective view of an embodiment of a spinal stabilization system;

FIGS. 2A and 2B depict perspective views of embodiments of a bone fastener;

FIG. 3A depicts a side cross-section view of a portion of one embodiment of a collar coupled to a bone screw;

FIG. 3B depicts a perspective view of one embodiment of a collar;

FIGS. 4-6 depict perspective views of embodiments of a collar;

FIGS. 7A and 7B depict side and top views of a portion of one embodiment of a spine stabilization system;

FIGS. 8A and 8B depict side and top views of the embodiment of a spine stabilization system depicted in FIGS. 7A and 7B;

FIGS. 9A and 9B depict side and top views of the embodiment of a spine stabilization system depicted in FIGS. 7A and 7B;

FIG. 10A depicts a cross section view of one embodiment of a portion of a spine stabilization system;

FIG. 10B depicts a close up partial view of the embodiment depicted in FIG. 10A;

FIG. 10C depicts a close up partial view of the embodiment depicted in FIG. 10A;

FIGS. 11A-11C depict side views of a portion of one embodiment of a spine stabilization system;

FIG. 12 depicts a side view of one embodiment of a portion of a spine stabilization system; and

FIG. 13 depicts a perspective view of a plunger useful in a spine stabilization system; and

FIG. 14 depicts a side exploded view of a portion of one embodiment of a spine stabilization system.

While the disclosure is susceptible to various modifications and alternative forms, specific embodiments thereof are shown by way of example in the drawings and will herein be described in detail. The drawings may not be to scale. It should be understood that the drawings and detailed description thereto are not intended to limit the disclosure to the particular form disclosed, but on the contrary, the intention is to cover all modifications, equivalents, and alternatives falling within the spirit and scope of the present disclosure as defined by the appended claims.

### DETAILED DESCRIPTION

A spinal stabilization system may be installed in a patient to stabilize a portion of a spine. Spinal stabilization may be used, but is not limited to use, in patients having degenerative disc disease, spinal stenosis, spondylolisthesis, pseudoarthrosis, and/or spinal deformities; in patients having fracture or other vertebral trauma; and in patients after tumor resection. A spinal stabilization system may be installed using a minimally invasive procedure. An instrumentation set may include instruments and spinal stabilization system components for forming a spinal stabilization system in a patient.

A minimally invasive procedure may be used to limit an amount of trauma to soft tissue surrounding vertebrae that are to be stabilized. In some embodiments, the natural flexibility of skin and soft tissue may be used to limit the length and/or depth of an incision or incisions needed during the stabilization procedure. Minimally invasive procedures may provide limited direct visibility in vivo. Forming a spinal stabilization system using a minimally invasive procedure may include using tools to position system components in the body.

A minimally invasive procedure may be performed after installation of one or more spinal implants in a patient. The spinal implant or spinal implants may be inserted using an anterior procedure and/or a lateral procedure. The patient may be turned and a minimally invasive procedure may be used to install a posterior spinal stabilization system. A minimally invasive procedure for stabilizing the spine may be performed without prior insertion of one or more spinal implants in some patients. In some patients, a minimally invasive procedure may be used to install a spinal stabilization system after one or more spinal implants are inserted using a posterior spinal approach.

A spinal stabilization system may be used to achieve rigid pedicle fixation while minimizing the amount of damage to surrounding tissue. In some embodiments, a spinal stabilization system may be used to provide stability to two adjacent vertebrae (i.e., one vertebral level). A spinal stabilization system may include two bone fastener assemblies. One bone fastener assembly may be positioned in each of the vertebrae to be stabilized. A rod may be coupled and secured to the bone fastener assemblies. As used herein, “coupled” components may directly contact each other or may be separated by one or more intervening members. In some embodiments, a single spinal stabilization system may be installed in a patient. Such a system may be referred to as a unilateral, single-level stabilization system or a single-level, two-point stabilization system. In some embodiments, two spinal stabilization systems may be installed in a patient on opposite sides of a spine. Such a system may be referred to as a bilateral, single-level stabilization system or a single-level, four-point stabilization system.

In some embodiments, a spinal stabilization system may provide stability to three or more vertebrae (i.e., two or more vertebral levels). In a two vertebral level spinal stabilization system, the spinal stabilization system may include three bone fastener assemblies. One bone fastener assembly may be positioned in each of the vertebrae to be stabilized. A rod may be coupled and secured to the three bone fastener assemblies. In some embodiments, a single two-level spinal stabilization system may be installed in a patient. Such a system may be referred to as a unilateral, two-level stabilization system or a two-level, three-point stabilization system. In some embodiments, two three-point spinal stabilization systems may be installed in a patient on opposite sides of a spine. Such a system may be referred to as a bilateral, two-level stabilization system or a two-level, six-point stabilization system.

In some embodiments, combination systems may be installed. For example, a two-point stabilization system may be installed on one side of a spine, and a three-point stabilization system may be installed on the opposite side of the spine. The composite system may be referred to a five-point stabilization system.

Minimally invasive procedures may reduce trauma to soft tissue surrounding vertebrae that are to be stabilized. Only a small opening may need to be made in a patient. For example, for a single-level stabilization procedure on one side of the spine, the surgical procedure may be performed through a 2 cm to 4 cm incision formed in the skin of the patient. In some embodiments, the incision may be above and substantially between the vertebrae to be stabilized. In some embodiments, the incision may be above and between the vertebrae to be stabilized. In some embodiments, the incision may be above and substantially halfway between the vertebrae to be stabilized. Dilators, a targeting needle, and/or a tissue wedge may be used to provide access to the vertebrae to be stabilized without the need to form an incision with a scalpel through muscle and other tissue between the vertebrae to be stabilized. A minimally invasive procedure may reduce an amount of post-operative pain felt by a patient as compared to invasive spinal stabilization procedures. A minimally invasive procedure may reduce recovery time for the patient as compared to invasive spinal procedures.

Components of spinal stabilization systems may be made of materials including, but not limited to, titanium, titanium alloys, stainless steel, ceramics, and/or polymers. Some components of a spinal stabilization system may be autoclaved and/or chemically sterilized. Components that may not be autoclaved and/or chemically sterilized may be made of sterile materials. Components made of sterile materials may be placed in working relation to other sterile components during assembly of a spinal stabilization system.

Spinal stabilization systems may be used to correct problems in lumbar, thoracic, and/or cervical portions of a spine. Various embodiments of a spinal stabilization system may be used from the C1 vertebra to the sacrum. For example, a spinal stabilization system may be implanted posterior to the spine to maintain distraction between adjacent vertebral bodies in a lumbar portion of the spine.

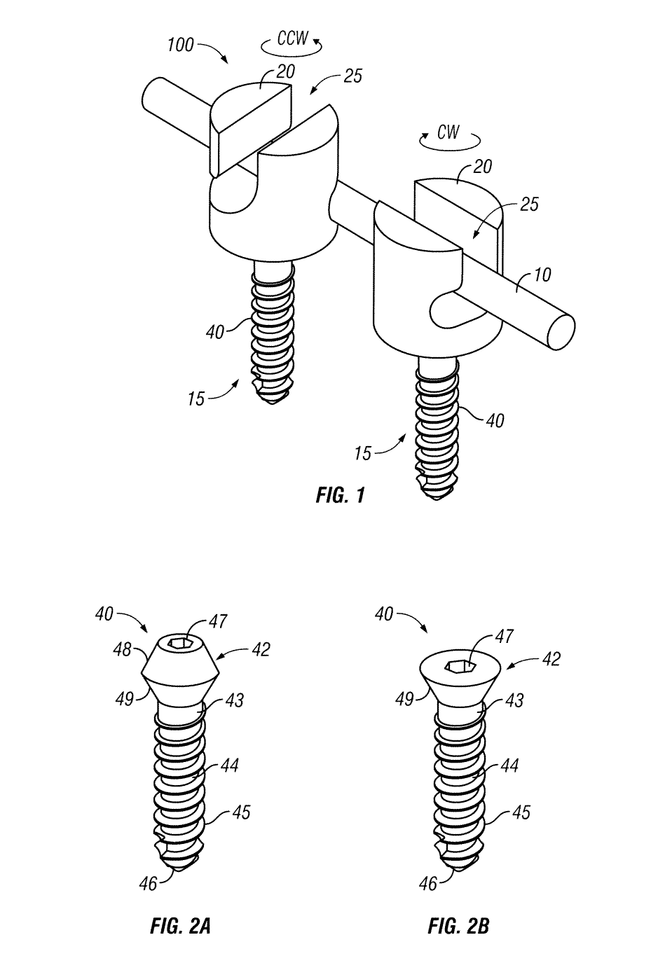

FIG. 1 depicts a perspective view of one embodiment of spinal stabilization system 100. In some embodiments, spine stabilization system 100 includes rod 10 having a length for spanning between two or more vertebrae, a plurality of bone fasteners 40 and collars 20. As used herein, the term “collar” includes any element that wholly or partially encloses or receives one or more other elements. Collar 20 may enclose or receive elements including, but not limited to, bone fastener 40 and/or rod 10. In some embodiments, collar 20 may couple two or more other elements together. In some embodiments, collar 20 may couple rod 10 and bone fastener 40 together. Collar 20 may have any of various physical forms.

In some embodiments, bone fastener 40 and collar 20 may be partially assembled to form bone fastener assembly 15. Partially assembling bone fastener 40 and collar 20 into bone fastener assembly 15 may be advantageous for reducing the complexity of the surgery, reducing the surgery time, reducing the risk of assembled components separating during surgery, and the like. After bone fastener 40 has been advanced into bony tissue and collar 20 has been positioned on bone fastener 40, rod 10 may be positioned in channel 25 of collar 20. Collar 20 may be rotated clockwise (CW) or counterclockwise (CCW) some angle to advance rod 10 in collar 20 and lock collar 20, rod 10 and bone fastener 40. In FIG. 1, a first collar 20 is depicted after counter-clockwise rotation to lock rod 10 in channel 25 and a second collar 20 is depicted after clockwise rotation to allow rod 10 to be inserted or withdrawn from collar 20. Locking collar 20, rod 10 and bone fastener 40 may provide rigid stabilization of a portion of the spine.

FIGS. 2A and 2B depict perspective views of embodiments of bone fastener 40. In some embodiments, each bone fastener 40 comprises head 42 and threaded shank 44 for advancement into the two or more vertebrae. In some embodiments, bone fastener 40 may include shank 44, head 42, and neck 43. Head 42 may include first surface 49. In some embodiments, head 42 may include first surface 49 and second surface 48. Shank 44 may include threading 45. In some embodiments, threading 45 may include self-tapping start 46. Self-tapping start 46 may facilitate insertion of bone fastener 40 into vertebral bone.

In some embodiments, head 42 may include surfaces 48 and 49 for contact with collar 20. In some embodiments, surfaces 48 and/or 49 of head 42 may be angles or curved. In some embodiments, head 42 of bone fastener 40 may include various configurations to engage a driver that inserts bone fastener 40 into a vertebra. In some embodiments, the driver may also be used to remove an installed bone fastener 40 from a vertebra. In some embodiments, head 42 may include one or more tool portions 47. Tool portions 47 may be recesses and/or protrusions designed to engage a portion of the driver. In some embodiments, bone fastener 40 may be cannulated for use in a minimally invasive procedure.

Bone fasteners 40 and/or bone fastener assemblies 15 may be provided in various lengths in an instrumentation set to accommodate variability in vertebral bodies. For example, an instrumentation set for stabilizing vertebrae in a lumbar region of the spine may include bone fastener assemblies 15 with lengths ranging from about 20 mm to about 75 mm in 5 mm increments. Bone fastener assembly 15 may be stamped with indicia (i.e., printing on a side of collar 20). In some embodiments, bone fastener assembly 15 or bone fastener 40 may be color-coded to indicate a length of bone fastener 40. In certain embodiments, bone fastener 40 with a 20 mm thread length may have a magenta color, bone fastener 40 with a 35 mm thread length may have an orange color, and bone fastener 40 with a 55 mm thread length may have a blue color. Other colors may be used as desired.

Each bone fastener 40 provided in an instrumentation set may have substantially the same thread profile and thread pitch. In some embodiments, thread 45 may have about a 4 mm major diameter and about a 2.5 mm minor diameter with a cancellous thread profile. In certain embodiments, the minor diameter of thread 45 may be in a range from about 1.5 mm to about 4 mm or larger. In certain embodiments, the major diameter of thread 45 may be in a range from about 3.5 mm to about 6.5 mm or larger. Bone fasteners 40 with other thread dimensions and/or thread profiles may also be used. A thread profile of bone fasteners 40 may allow bone purchase to be maximized when bone fastener 40 is positioned in vertebral bone.

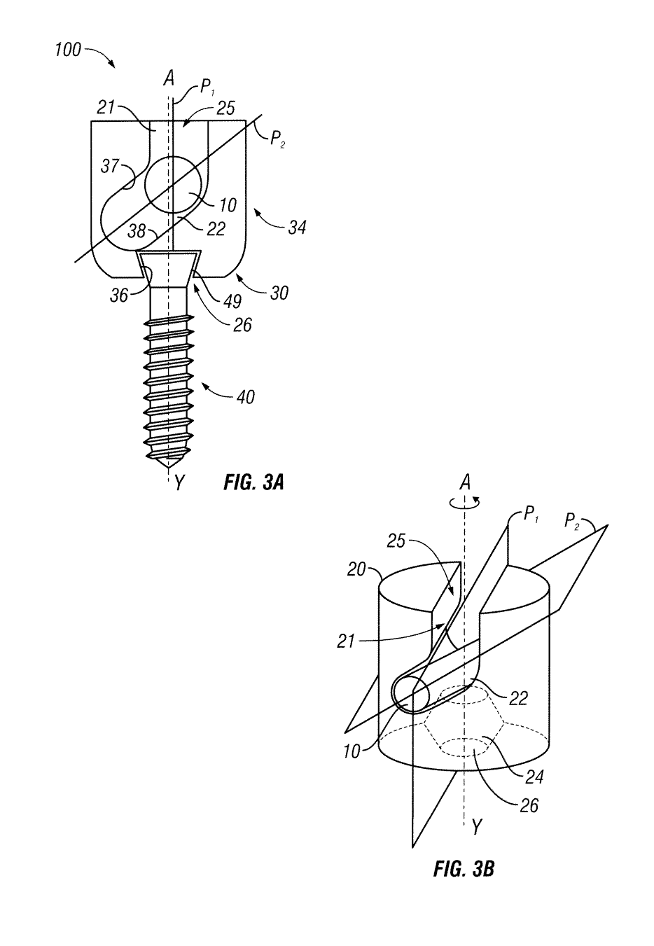

FIGS. 3A and 3B depict side and perspective views of one embodiment of collar 20. In some embodiments, collar 20 has first end 30 and second end 34. In some embodiments, first end 30 has opening 26 for accommodating shank 44 of bone fastener 40 and cavity 24 recessing from opening 26 into first end 30 for accommodating head 42 of bone fastener 40.

In some embodiments, second end 34 includes channel 25 for receiving rod 10. Channel 25 may be sized to receive rod 10. Channel 25 may include, but is not limited to, an elongated opening of constant width, an elongated opening of variable width, a rectangular opening, a trapezoidal opening, a circular opening, a square opening, an ovoid opening, an egg-shaped opening, a tapered opening, and combinations and/or portions thereof. In some embodiments, first portion 21 of channel 25 may have different dimensions than second portion 22 of channel 25. When rod 10 is positioned in channel 25, a portion of rod 10 may contact head 42 of bone fastener 40 positioned in cavity 24 of collar 20.

In FIG. 3A, collar 20 is depicted as having first portion 21 of channel 25 in first plane P₁. In some embodiments, plane P₁ may intersect with axis AY of bone fastener 40. In some embodiments, first portion 21 may lie in plane P₁ oriented at some angle to axis AY. In some embodiments, first portion 21 may lie in plane P₁ oriented substantially perpendicular to axis AY. In some embodiments, first portion 21 may lie in plane P₁ such that axis AY lies in plane P₁. In some embodiments, second portion 22 of channel 25 may lie in a second plane P₂. Second plane P₂ may define a linear path or a curvilinear path for rod 10. In some embodiments, second plane P₂ may be defined at some angle relative to first plane P₁.

Rod 10 may be positioned in channel 25 and advanced along first portion 21. In some embodiments, channel 25 includes first portion 21 having a first interior geometric configuration to allow rod 10 to move relative to collar 20. In some embodiments, first portion 21 may have a first geometric configuration to allow rod 10 to be moved freely within channel 25. In some embodiments, first portion 21 may include a geometric configuration including textured surfaces, protuberances, or tapered sides to provisionally secure rod 10 in channel 25. Provisionally locking rod 10 in collar 20 may allow a surgeon to make changes to spine stabilization system 100 or implant other components of spine stabilization system 100 before securely locking rod 10 in collar 20.

In some embodiments, rod 10 may be advanced into second section 22. In some embodiments, collar 20 may be rotated about axis AY to advance rod 10 into second section 22. In some embodiments, rotation of collar 20 may be counter-clockwise. In some embodiments, rotation of collar 20 may be clockwise. In some embodiments, rotation of collar 20 may comprise less than 90 degrees. In some embodiments, rotation of collar 20 may comprise less than 45 degrees. In some embodiments, rotation of collar 20 may comprise less than 30 degrees. In some embodiments, rotation of collar 20 may comprise less than 20 degrees. In some embodiments, rotation of collar 20 may comprise less than 10 degrees. In some embodiments, rotation of collar 20 about axis AY may advance rod 10 along plane P₂ toward a second position. In some embodiments, rotation of collar 20 may advance rod 10 into second portion 22 of channel 25 to contact bone fastener 40. In some embodiments, rotation of collar 20 may bias a first side 37 of channel 25 against rod 10. In some embodiments, rotation of collar 20 with rod 10 in contact with bone fastener 40 may bias a first side 36 of cavity 24 against surface 49 of bone fastener 40. In some embodiments, rotation of collar 20 with rod 10 in contact with bone fastener 40 may bias a second side 38 of channel 25 against rod 10. In some embodiments, biasing first side 37 of channel 25 against rod 10 may cause cold welding of bone fastener 40 to collar 20.

In some embodiments, rotating collar 20 about its longitudinal axis may cause rod 10 to contact head 42 of bone fastener 40. In some embodiments, contact between rod 10 and bone fastener 40 may inhibit further advancement or movement of rod 10. In some embodiments, continued rotation of collar 20 may bias collar 20 to contact bone fastener 40 such that surface 48 or 49 of head 42 of bone fastener 40 may contact surface 36 or 39 of cavity 24. In some embodiments, continued rotation of collar 20 may cause cold welding between surface 48 or 49 of head 42 and surface 36 or 39 of cavity 24.

In some embodiments, continued rotation of collar 20 may cause cold welding between rod 10 and head 42 of bone fastener 40. In some embodiments, cold welding may inhibit movement of one component relative to another component. In some embodiments, cold welding may inhibit movement between collar 20 and bone fastener 40. In some embodiments, cold welding may inhibit movement between collar 20 and rod 10. In some embodiments, cold welding may inhibit movement between rod 10 and bone fastener 40.

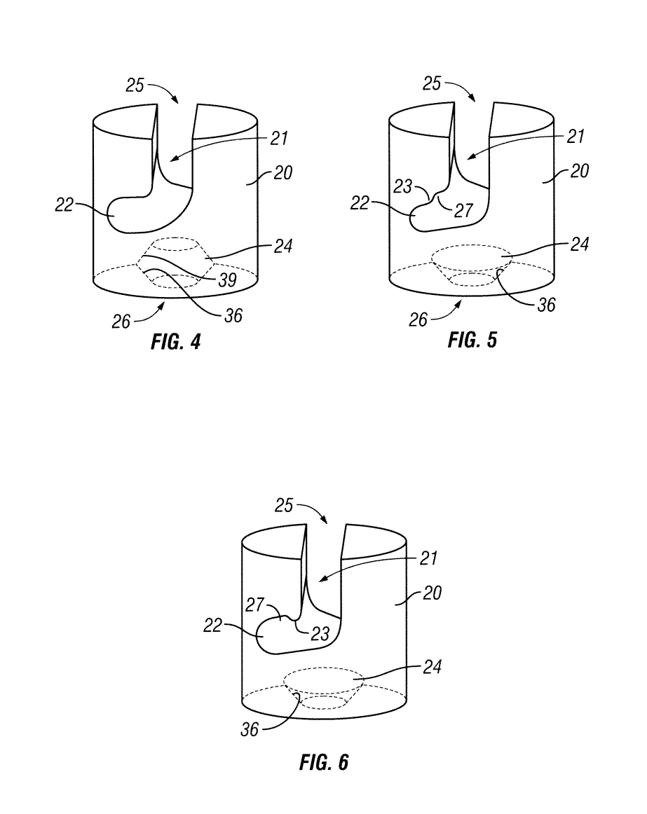

FIGS. 4-6 depict embodiments of collar 20 in which cavity 24 may be formed into different shapes or profiles for contact with heads 42 of bone fasteners 42, and in which channel 25 may be formed in different planes. In some embodiments, cavity 24 may be formed with surface 36 or 39 for contact with head 42 of bone fastener 40. FIG. 4 depicts one embodiment in which cavity 24 of collar 20 has surfaces 36 and 39 for contact with head 42 of bone fastener 40. FIGS. 5 and 6 depicts embodiments in which depicts one embodiment in which cavity 24 of collar 20 has surface 36 for contact with head 42 of bone fastener 40.

In some embodiments, second portion 22 of channel 25 may lie in a plane or may have a curvilinear path. FIG. 4 depicts one embodiment of collar 20 having second section 22 of channel 25 formed into a circular helix shape. Rod 10 may be positioned in first portion 21 of channel 25 and collar 20 may be rotated to advance rod 10 based on the curvilinear path of second portion 22.

FIGS. 5 and 6 depict collar 20 having second portion 22 of channel 25 oriented on a plane. FIGS. 5 and 6 depict embodiments in which second portion 22 of channel 25 may include protuberance 23 and recessed portion 27. FIG. 5 depicts one embodiment in which recessed portion 27 in second portion 22 of channel 25 may allow rod 10 to be provisionally locked in collar 20. Further rotation of collar 20 may bias rod 10 inferior to protuberance 23 to provide higher resistance between rod 10 and collar 20. In some embodiments, when rod 10 is in recessed portion 27, some cold welding may occur, and when rod 10 is positioned inferior to protuberance 23, additional cold welding occurs.

FIG. 6 depicts one embodiment in which second portion 22 of channel 25 includes protuberance 23 and recessed portion 27. In some embodiments, when rod 10 is positioned inferior to protuberance 23, cold welding may occur between components in spine stabilization system 100, and when rod 10 is positioned in recessed portion 27, protuberance 23 may provide lateral or tangential resistance on rod 10 to inhibit withdrawal of rod 10 from channel 25.

Instruments used to install a spinal stabilization system may be made of materials including, but not limited to, stainless steel, titanium, titanium alloys, ceramics, and/or polymers. Some instruments may be autoclaved and/or chemically sterilized. Some instruments may include components that cannot be autoclaved or chemically sterilized. Components of instruments that cannot be autoclaved or chemically sterilized may be made of sterile materials. The sterile materials may be placed in working relation to other parts of the instrument that have been sterilized.

A targeting needle may be used to locate an entry point in a vertebral body for a bone fastener of a bone fastener assembly. In some embodiments, the targeting needle may be a Jamshidi® bone marrow biopsy needle. A targeting needle may include an outer housing including a hollow shaft and a handle. Scale markings may be printed, etched, or otherwise placed on a hollow shaft. Scale markings may be used to approximate a length of a bone fastener needed for a vertebra. A handle may provide a grip that allows a user to manipulate the targeting needle. A handle may include a threaded portion for coupling to threading on a portion of a targeting needle member to secure the member to the outer housing.

In some embodiments, a guide wire may pass down a shaft of a targeting needle outer housing. A guide wire may be from about 15 cm to about 65 cm in length. In some embodiments, guide wires provided in an instrumentation set are about 46 cm in length. The length of a guide wire may allow a surgeon and/or assistants to hold at least one portion of the guide wire at all times when the guide wire is inserted into vertebral bone, even during insertion, use, and removal of instruments along a length of the guide wire. A guide wire that can be held continuously during a surgical procedure may inhibit removal or advancement of the guide wire from a desired position during a minimally invasive surgical procedure.

In some embodiments, a distal end of a guide wire may include a point. A point may facilitate insertion of the distal end of the guide wire into vertebral bone. In some embodiments, a distal end of a guide wire may not be pointed. A position of an unpointed guide wire in bone may be easier to maintain during a spinal stabilization procedure.

Dilators may be used during a minimally invasive surgical procedure to push aside tissue and create space to access vertebral bone. In some embodiments, four tissue dilators of increasing diameter may be used to establish sufficient working space to accommodate instruments and spinal stabilization system components. In some embodiments, especially for a mid-vertebra or for mid-vertebrae of a multi-level stabilization system, only three dilators may be needed to form sufficient working space. Dilators in an instrumentation set may increase in diameter incrementally by a selected amount. For example, outside diameters of dilators in an instrumentation set may increase sequentially by increments of about 0.5 mm.

A bone awl may be used to breach cortical bone of a pedicle. In some embodiments, a bone awl may include a handle, a passage, and a tip. The handle may provide a secure grip that allows a surgeon to breach cortical bone of a pedicle with the tip. A guide wire that is inserted in vertebral bone in a desired orientation may be inserted through a passage that extends through the bone awl. A bone awl may be moved down the guide wire so that the tip contacts the pedicle.

In some embodiments, a bone awl may have a length that allows a guide wire positioned in vertebral bone to always be held in at least one location when the guide wire is placed through a passage in the needle. In some embodiments, the handle may be removable from a shaft of a bone awl so that the guide wire may always be held during use of the bone awl.

During some surgical procedures downward force and some rotation of the bone awl may be sufficient to breach cortical bone of a vertebra. During some surgical procedures, an impact force may be needed for the bone awl to breach cortical bone. In some embodiments, a guide wire may be removed, the bone awl may be used to breach cortical bone, and the guide wire may be reinserted. In some embodiments, a small dilator may be placed over the portion of the guide wire extending from the bone awl so that a first end of the dilator contacts the bone awl. A mallet or other impact device may be used against a second end of the dilator so that the bone awl breaches cortical bone of the vertebra. The dilator may be removed from the bone awl and contact with the guide wire may be reestablished.

A bone tap may be used to form a threaded passage of a desired depth through a pedicle and into a vertebral body. In some embodiments, a tap may include a passage, a shaft, a removable handle, flutes, and indicia. The passage may extend through a length of the shaft and the removable handle. A guide wire positioned in vertebral bone may be inserted into a distal end of the passage so that the tap can be moved down the guide wire toward the bone. In some embodiments, a proximal portion of the shaft may include at least one flat portion that fits in a mating portion of a removable handle. The proximal end of the shaft may also include a detent depression. The flat portion may allow for rotation of the shaft when the removable handle is rotated. An embodiment of the removable handle may include a spring-loaded release. When the spring-loaded release is compressed, a detent in the removable handle may be movable. When the spring-loaded release is not compressed, movement of the detent may be inhibited. When the shaft is positioned in the removable handle, the detent of the removable handle may be positioned in the detent depression of the shaft to couple the shaft to the removable handle.

In some embodiments, x-ray monitoring of a depth of a tap portion of known length may allow a medical practitioner to assess a depth of a hole tapped in a bone. In some embodiments, the hole may be tapped to accommodate a bone fastener of a desired length. In certain embodiments, a bone fastener may be chosen to accommodate a hole tapped to a desired depth.

A guide wire positioned in vertebral bone may be held near a top of a dilator inserted over the guide wire at a surgical site. A proximal end of the guide wire may be positioned through a distal end of a passage in the shaft of the tap without a removable handle coupled to the shaft. A proximal portion of the guide wire may be held when the proximal portion of the guide wire extends beyond the top of the shaft. A portion of the guide wire may always be held during use of the tap. The shaft may be moved down the guide wire until the shaft contacts the vertebral bone. The guide wire may be held near the top of the shaft and the guide wire may be positioned through the passage of the removable handle. When the guide wire extends out of the passage through the removable handle, the guide wire may be held above the removable handle. The handle may be coupled to the shaft using a spring-loaded release.

In some embodiments, a first reading of indicia relative to a proximal end of a dilator may be taken when one or more flutes are located at a pedicle. In some embodiments, the tap may be rotated so that the flutes form a threaded opening through the pedicle and into a vertebral body. The flutes may have a diameter that is about 0.1 mm to about 0.7 mm less than a maximum thread flight of a bone fastener to be positioned in the threaded opening formed by the flutes. In one embodiment, the tap may form a thread that is about 0.5 mm less than a maximum thread flight of a bone fastener to be positioned in the threaded opening formed by the flutes. In some embodiments, a position of the tap may be monitored using a fluoroscope. In some embodiments, when the threaded opening is formed to a desired depth, a second reading of the indicia relative to the dilator may be taken. In some embodiments, a length of a bone fastener to be inserted into the vertebral body may be estimated by taking the difference between the indicia readings.

In some embodiments, after a threaded opening is formed to a desired depth, the tap may be removed by rotating the tap until the flutes are disengaged from vertebral bone. A removable handle may be separated from the shaft, and the removable handle may be removed with the guide wire always held in at least one location. After the removable handle is removed from the guide wire, the shaft may be removed with the guide wire always held in at least one location.

A detachable member may be used as a guide to install bone fasteners of a bone fastener assembly in vertebral bone. A detachable member may be coupled to a collar of a bone fastener assembly. A distal end of a detachable member may be tapered or angled to reduce bulk at a surgical site. Instruments may be inserted into the detachable member to manipulate the bone fastener assembly. Movement of the detachable member may alter an orientation of a collar relative to a bone fastener of the bone fastener assembly. In some embodiments, a detachable member may be used as a retractor during a spinal stabilization procedure.

A detachable member for a single-level vertebral stabilization system may include one or more channels in a wall of the detachable member to allow access to an adjacent vertebra. For some single-level vertebral stabilization procedures, only single-channel detachable members (i.e., detachable members with a single channel in a wall of the detachable member) may be used. For other single-level vertebral stabilization procedures, one or more multi-channel detachable members (i.e., detachable members with two or more channels in a wall of the detachable member) may be used. In some embodiments, channels may provide flexibility to or enhance flexibility of a multi-channel detachable member. In some embodiments, a proximal portion of a multi-channel detachable member may have a solid circumference. In some embodiments, a region of solid circumference in a multi-channel detachable member may enhance stability of the multi-channel detachable member. In some embodiments, a multi-channel detachable member may be longer than a single-channel detachable member.

In some embodiments, a detachable member used at a middle vertebra in a multi-level stabilization procedure may be a multi-channel detachable member. In some embodiments, channels in a multi-channel detachable member may allow access to adjacent vertebrae from a middle vertebra. In some embodiments, a detachable member used at an end vertebra of a multi-level stabilization system may be a single-channel detachable member or a multi-channel detachable member. In some embodiments, a system for coupling a bone fastener assembly to a multi-channel detachable member may include a limiter that inhibits spreading of arms of the detachable member to inhibit release of the bone fastener assembly from the detachable member.

In some embodiments, a channel in a wall of a detachable member may allow access to a vertebra that is to be stabilized with a spinal stabilization system being formed. In some embodiments, a single-channel detachable member may be coupled to a bone fastener assembly to be inserted into vertebral bone of a first vertebra. The single-channel detachable member may allow access to a second vertebra from the first vertebra. In other embodiments, a multi-channel detachable member may be coupled to a bone fastener assembly to be inserted into vertebral bone of a first vertebra. The multi-channel detachable member may allow access from the first vertebra to adjacent vertebrae.

Instruments may access a bone fastener assembly through a passage in a detachable member. In some embodiments, a channel in a wall of a detachable member may extend a full length of the detachable member. In some embodiments, especially in embodiments of multi-channel detachable members, a channel in a wall of a detachable member may extend only a portion of the length of the detachable member. In some embodiments, a channel in a wall of a detachable member may extend 25%, 50%, 75%, 80%, 90%, 95% or more of the length of the detachable member. A channel may extend to a distal end of a detachable member such that a rod inserted in the channel may pass from the detachable member into a slot of a collar of a bone fastener assembly coupled to the detachable member.

A channel in a detachable member may be any of a variety of shapes. A channel may have a width that exceeds a width (e.g., a diameter) of a rod that is to be inserted in the channel. In some embodiments, a channel may be a linear opening parallel to a longitudinal axis of the detachable member. In some embodiments, a channel may have a non-linear shape including, but not limited to, a helical pattern, an arc, an “L” shape, or an “S” shape. A non-linear channel may allow a rod to travel along a predetermined path. In certain embodiments, adjacent detachable members may include channels with matching profiles, allowing ends of a rod to follow similar paths down the detachable member channels.

Movable members may extend through portions of a detachable member proximate a channel in the detachable member. Movable members may engage notches in a collar to establish a radial orientation of the detachable member on the collar and/or to inhibit rotation of the collar relative to the detachable member. A distal end of a movable member may be flat, curved, or angled. In some embodiments, a distal end of a movable member may be threaded. In other embodiments, a distal end of a movable member may be a projection that engages an opening in a collar. In some embodiments, an upper surface of a collar and/or a surface of a distal end of a movable member may be textured to inhibit rotation of the collar relative to the detachable member. In certain embodiments, a proximal end of a movable member may include a tool engaging portion. A tool engaging portion may include, but is not limited to, a hex section, a hexalobular section, a tapered section, a bead, a knot, a keyed opening, a coating, a threading, and/or a roughened surface for engaging a drive that rotates or otherwise displaces the movable member.

A cross section transverse to a longitudinal axis of a detachable member may have shapes including, but not limited to, circular, ovoid, square, pentagonal, hexagonal, and combinations thereof. In some embodiments, a detachable member may be hollow. In certain embodiments, a thickness of a hollow detachable member may be uniform. In certain embodiments, a thickness of a hollow detachable member may vary along the length of the detachable member. A detachable member with a passage extending longitudinally from a first end of the detachable member to a second end of the detachable member may be referred to as a “sleeve”.

In some embodiments, a sleeve may be a multi-channel sleeve. In some embodiments, a sleeve may include a wall, channels, a passage, movable members, and a flange. In some embodiments, channels may extend from a distal end of the sleeve through a portion of the wall. In some embodiments, channels may allow instruments to be positioned and used to form a plane through soft tissue to one or more adjacent vertebrae. A rod may be inserted in the tissue plane and positioned in collars of bone fastener assemblies anchored in vertebrae and coupled to sleeves. In some embodiments, a passage may allow instruments to be positioned and used to manipulate a bone fastener assembly that is coupled to a distal end of the sleeve. Movable members may be part of a system that couples a bone fastener assembly to a sleeve. In some embodiments, movable members may include a tool engaging portion. A driver may be positioned in the tool portion. The driver (e.g., a hex wrench) may be used to extend or retract a distal end of a movable member. A distal end of a sleeve may include a flange that mates with a complementary flange on a collar of a bone fastener assembly. A distal end of a sleeve may be tapered to reduce bulk at a surgical site.

In some embodiments, a flange of a sleeve may mate with a flange of a collar to inhibit translation of the sleeve relative to the collar. In some embodiments, a sleeve may also include a stop. In some embodiments, the stop may engage a portion of a collar to inhibit separation of the walls. During use, a stop may inhibit undesired separation of a bone fastener assembly from a sleeve.

In some embodiments, distal ends of the movable members may extend into notches in the collar. Portions of the walls of a sleeve may include threading. Portions of the movable members may include threading complementary to threaded portions of the walls. Threading of the movable members may engage threading in the walls such that rotation of the movable members advances or retracts the movable members relative to the walls.

In some embodiments, a collar may be designed such that a rod lies below a distal end of a sleeve. In some embodiments, coupling a sleeve to a collar above a rod may reduce bulk at a surgical site. With a rod coupled to a collar below a distal end of a sleeve, the sleeve may be removed without interference from the rod of a spinal stabilization system.

In some embodiments, a sleeve may be a single-channel sleeve for use in single-level or multi-level spinal stabilization procedures. In some embodiments, a sleeve may be used at the outermost vertebrae to be stabilized during installation of a multi-level vertebral stabilization system. In some embodiments, a sleeve may be coupled to a collar of a bone fastener assembly with movable members and/or a flange. In some embodiments, instruments may be inserted through a passage of a sleeve to access an anchored bone fastener assembly coupled to the sleeve. In some embodiments, an instrument may be moved through a channel toward an adjacent vertebra to form a tissue plane in soft tissue between the sleeve and the adjacent vertebra.

A sleeve may be coupled to a bone fastener assembly in various ways to inhibit movement of the sleeve relative to a collar of the bone fastener assembly. A system used to couple the sleeve to the bone fastener assembly may inhibit rotation and translation of the sleeve relative to the collar.

In some embodiments, a sleeve may include movable members to inhibit removal of the sleeve from the collar. In some embodiments, movable members may include threaded distal end portions. In some embodiments, a collar may include openings. In some embodiments, the openings may be threaded. In some embodiments, the openings of a collar may be aligned with the movable members. In some embodiments, a drive end of a driver may be positioned in a tool engaging portion of the movable member. In some embodiments, a driver may be rotated to couple a threaded end of a movable member with threads in an opening. In some embodiments, the driver may be positioned in a tool opening of a second movable member. In some embodiments, the driver may be used to couple a threaded end of a second movable member with threads in a second opening. Threaded connections between the movable members and the collar may inhibit movement of the collar relative to the sleeve.

A detachable member may be coupled to a collar of a bone fastener assembly in various ways. When a detachable member is coupled to a collar, rotation and translation of the detachable member relative to the collar may be inhibited. A system used to couple a detachable member and collar should be simple, inexpensive to implement, and should not significantly weaken the mechanical strength of the collar and/or the detachable member. Detachable members may be coupled to collars using various coupling systems including, but not limited to, flanges, threaded connections, interlocking connections (e.g., ratcheting connection systems), and/or interference fits.

In one embodiment of an interlocking connection system, a detachable member may include an opposing pair of deflectable arms. Each deflectable arm may include a tooth. The deflectable arms may be forced outwards during coupling of a collar to the detachable member. When the collar is coupled to the detachable member, the deflectable arms may be positioned in channels in the collar, with the teeth positioned in indentions in the collar. The presence of the deflectable arms in the channels of the collar may inhibit rotation and translation of the detachable member relative to the collar. Separation of the detachable member from the collar may be achieved by insertion of an expander in the detachable member. The expander may be used to force the deflectable arms outwards and expel the teeth from the indentions.

In some embodiments, a rod advanced in the collar of the bone fastener assembly would lie below a distal end of a sleeve. Having the rod below the distal end of a sleeve reduces bulk at the surgical site. With a sleeve positioned above the rod, interference of the secured rod with the sleeve is avoided during removal of the sleeve.

In some embodiments, the detachable member and the collar may include members that work together to inhibit radial expansion of walls of the detachable member. In some embodiments, a stop in a sleeve and a ledge in a collar may be needed in a multi-channel sleeve embodiment. A stop in a sleeve and/or a ledge in a collar may not be needed in a single-channel sleeve embodiment or in a collar for a single-level stabilization.

In some detachable member and collar coupling embodiments, a detachable member may include a protrusion that mates with a complementary groove in a collar. Alternatively, a detachable member may include a groove that mates with a complementary protrusion of a collar.

In some embodiments, a detachable member and/or a collar may include a locking system to inhibit rotation of the detachable member relative to the collar. The locking system may be, but is not limited to, threading, interference fits, frictional engagement, or a press-fit connection. In some embodiments, a locking system may inhibit translation and/or rotation of a detachable member relative to a collar.

In one embodiment, an inner sleeve may be positioned in a sleeve to inhibit translation and/or rotation of the sleeve relative to a collar of a bone fastener assembly. In some embodiments, a distal end of an inner sleeve may contact an upper end of a collar. A proximal portion of an inner sleeve may engage a proximal portion of a sleeve. The engagement may allow the inner sleeve to apply a force against a collar that presses a flange against other flanges of sleeves to inhibit translation of the sleeve relative to the collar. The engagement may be, but is not limited to, a threaded connection, an interference fit, a frictional fit, or a keyway type of connection.

In some embodiments, a distal end of an inner sleeve may be roughened or textured to frictionally engage a proximal surface of the collar. The frictional engagement may inhibit rotation of the sleeve relative to the collar. In some embodiments, threading may be used to couple a detachable member to a collar. In some embodiments, threading of the sleeve and threading of the collar may be modified threads.

In some embodiments, a detachable member may include a pair of hinged arms configured to couple to a collar. In some embodiments, a sleeve may include arms. Arms may be pivotally coupled together by a hinge. In some embodiments, a hinge may be located near a proximal end of a sleeve. In some sleeve embodiments, a sleeve may include a locking element or a biasing element (e.g., a spring) near or at a hinge. A locking element or biasing element may cause a clamping force to be exerted on a collar to maintain the collar in the sleeve and/or to inhibit rotation of a collar in a sleeve.

In some embodiments, proximal portions of detachable members may be chamfered to allow ends of the detachable members to more closely approach each other than detachable members with a uniform cross section. In some embodiments, during some surgical procedures, only one of the sleeves may be chamfered. During some surgical procedures, the use of a sleeve with a chamfered surface may allow for a smaller incision than required when using non-chamfered sleeves. In some embodiments, other types of detachable members may be used to reduce space between proximal ends of detachable members. Other types of detachable members may include, but are not limited to, detachable members of different lengths, detachable members of different diameters, and detachable members with flexible end portions.

Detachable members may be of various lengths. Detachable members of different lengths may be used in the same surgical procedure. A detachable member length used in a spinal stabilization procedure may be determined by a patient's anatomy. Detachable members may be just short enough to allow manipulation by a medical practitioner above an incision in a patient. In some embodiments, detachable members may be about 3.5 to about 11.5 cm long. For example, a single-channel detachable member may be about 10 cm long. In some embodiments, detachable members may be about 11.5 cm to about 14 cm long. For example, a single-channel or a multi-channel detachable member may be about 12.5 cm long. A multi-channel detachable member may be longer than a single-channel detachable member. In some embodiments, a multi-channel detachable member may be at least about 15 cm long. For example, a multi-channel detachable member may be about 16 cm long. Detachable members that are too long may require a longer incision and/or a larger tissue plane for insertion of a spinal stabilization system. Insertion of a rod may be more difficult with detachable members that are longer than necessary. Detachable members with excess length may be bulky and hard to manipulate during a surgical procedure.

A detachable member may be flexible over its entire length or include a flexible portion near a proximal end of the detachable member. A flexible portion may allow positioning of a proximal portion of a detachable member in a desired location. A flexible portion may be produced from any of various materials including, but not limited to, a surgical grade plastic, rubber, or metal. A flexible portion may be formed of various elements, including, but not limited to, a tube, a channel, or a plurality of linked segments. During some spinal stabilization procedures, a detachable member without a second portion that is able to move relative to a first portion may be used at one vertebra, and a detachable member with a second portion that is able to move relative to a first portion may be used at one or more vertebrae that are to be stabilized.

When bone fasteners of bone fastener assemblies are positioned in vertebral bone, detachable members coupled to collars of the bone fastener assemblies may be moved in desired positions. During surgery, a detachable member in a patient may be oriented towards an adjacent vertebra that is to be stabilized to reduce the required incision size. In some embodiments, channels of the detachable members may be aligned so that a rod may be positioned in collars of the bone fastener assemblies. In some embodiments, sleeves may couple to collars. In some embodiments, bone fasteners may be inserted into vertebrae. In some embodiments, single-channel sleeves may be coupled to the collars before insertion of the bone fasteners into two outer pedicles to be stabilized. In some embodiments, a multi-channel sleeve may be coupled to a collar before insertion of a bone fastener into a central pedicle of the three adjacent pedicles. In some embodiments, single-channel sleeves may be angled towards a multi-channel sleeve. In certain embodiments, multi-channel detachable members may be coupled to all three pedicles. In other embodiments, differently shaped detachable members (e.g., circular, oval) may be used in one or more of the pedicles. Channels of the detachable members may be aligned so that a rod may be moved down the detachable members and into collars of the bone fastener assemblies.

In some embodiments, channels of detachable members may face a direction other than toward each other. In some embodiments, channels in the detachable member may not be longitudinal channels down the length of the detachable member. In embodiments of detachable members with non-longitudinal channels, the channels of two adjacent detachable members may not face towards each other when the openings of collars coupled to the detachable members are aligned.

In some embodiments, a sleeve is coupled to a bone fastener assembly. In some embodiments, a driver may be coupled to the collar and to the bone fastener of the bone fastener assembly. In some embodiments, coupling a driver to collar and to a bone fastener may ensure proper alignment of the driver relative to the bone fastener. In some embodiments, coupling a driver to a collar and to a bone fastener may also inhibit movement of the collar relative to the bone fastener during insertion of the bone fastener.

During a minimally invasive surgical procedure, a plane may be created in tissue from a first vertebra to a second vertebra. A rod may be positioned in the plane during the surgical procedure. In some embodiments, a tissue plane may be formed using a targeting needle. The targeting needle may be positioned at the first vertebra. The distal end of the needle may be moved toward the second vertebra to form the plane while maintaining a position of the needle at a surface of the skin. The needle may be moved back and forth a number of times to clearly establish the plane. Care may need to be taken to avoid bending the targeting needle during establishment of the plane.

In some embodiments, a tissue wedge may be used to form a plane in tissue between a first vertebra and a second vertebra. The blade may be a double-wedged blade. A blade may have a diamond-like shape. In some embodiments, the edges of a blade may be blunt to avoid severing tissue during use of a tissue wedge. In some embodiments, the distal end of the blade may be rounded. A shape of distal end may inhibit damage to tissue and may facilitate movement of the blade towards a target location during formation of a plane in tissue between vertebrae. In some tissue wedge embodiments, a tissue wedge may include a hook. The cutting edge in the hook may be used to sever portions of tissue (e.g., fascia) through which the blade cannot form a plane. In some embodiments, the cutting edge may be oriented in the blade so that severing of tissue results when the tissue wedge is pulled away from the spine.

An estimating tool may be used to estimate a distance between bone fastener assemblies anchored in vertebrae. The bone fastener assemblies may be part of a single-level or multi-level spinal stabilization system. The distance estimated by an estimating tool may be used to determine a desired length of a rod to be positioned in collars of the anchored bone fastener assemblies. In one embodiment, a length of a rod may be chosen to be greater than a distance between bone fastener assemblies to allow for bending of the rod and/or to allow the rod to extend beyond the collars of the anchored bone fastener assemblies. For example, 15 mm may be added to the distance between bone fastener assemblies. In some embodiments, a length of a rod may be chosen such that the rod extends 2 mm or more beyond the collars. In certain embodiments, a length of a rod may be chosen such that ends of the rod do not extend from the collars.

In some embodiments, an estimating tool may include a gage. With the arms of an estimating tool positioned together, a gage may have or may be set to a zero reading. With the arms extended to meet resistance in the sleeves, the gage may provide an estimate of the distance between the sleeves. The distance between the sleeves may be used to estimate a length of a rod needed to couple the anchored bone fastener assemblies. In one embodiment, a length of a rod may be chosen to be greater than the distance measured by a gage to allow the rod to extend beyond slots of collars of anchored bone fastener assemblies.

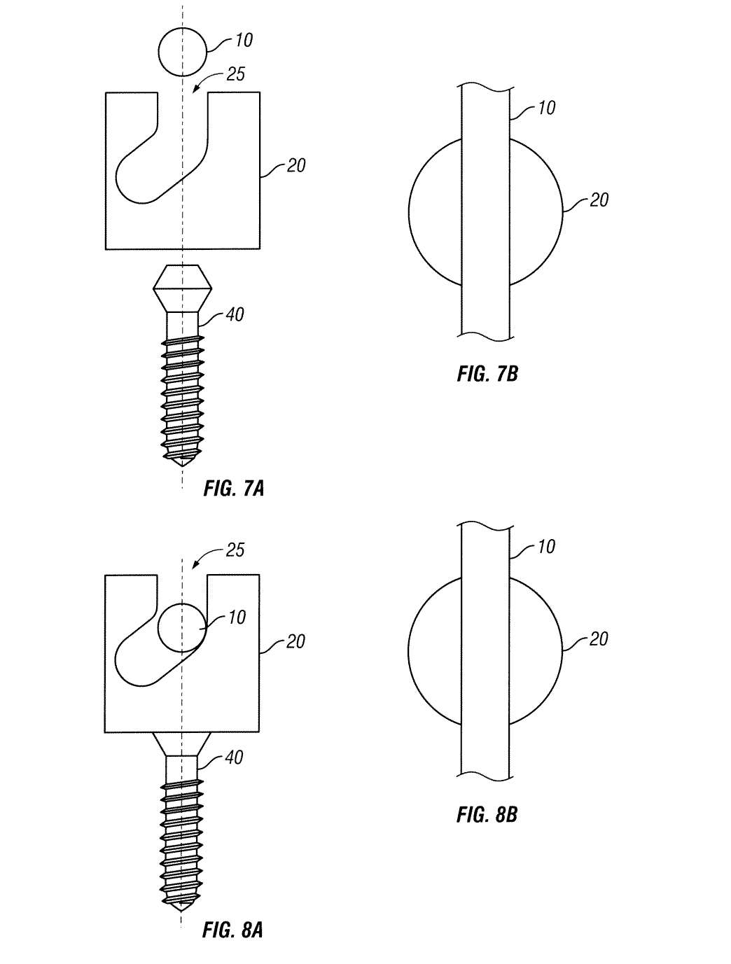

In some embodiments, components of spine stabilization system 100 may be advanced into a patient. FIGS. 7A and 7B depict side and top views of one embodiment of a portion of spine stabilization system 100 before rod 10 is positioned in collar 20. In some embodiments, bone fastener 40 may be advanced into a bone and collar 20 may be advanced onto bone fastener 40. In some embodiments, collar 20 and bone fastener 40 may be coupled outside the patient and advanced into the patient as bone fastener assembly 15. In some embodiments, channel 25 may be oriented substantially parallel with the spine. In some embodiments, rod 10 may be advanced into the patient and also oriented substantially parallel with the spine. In some embodiments, bone fastener 40, collar 20, bone fastener 15, and/or rod 10 may be advanced into the patient using invasive surgery techniques. In some embodiments, some or all components of spine stabilization system 100 may be advanced using Minimally Invasive Surgery (MIS) techniques. In some MIS techniques, bone fastener 40, collar 20, bone fastener assembly 15, or rod 10 may be advanced into the patient via sleeves or dilators (not shown).

In some embodiments, rod 10 may be positioned in channel 25 of collar 20. FIGS. 8A and 8B depict side and top views of one embodiment of spine stabilization system 100. As depicted in FIG. 8A, rod 10 may be positioned in first portion 21 of channel 25. In some embodiments, channel 25 may have a width such that rod 10 positioned in first portion 21 may be provisionally locked to collar 20. Provisionally locking rod 10 to collar 20 may allow a surgeon to modify spine stabilization system 100 or adjust the position, angle or orientation of components of spine stabilization system. In some embodiments, a rod positioner may be used to guide rod 10 through detachable members and to position rod 10 in first portion 21 of collar 25 proximate pedicles of vertebrae. In some embodiments, rod 10 may be coupled to the rod positioner. The distal end of the rod positioner may be contoured (e.g., curved) to allow some motion (e.g., rocking motion) of rod 10 while rod 10 is coaxed into first portion 21 of channel 25. During some installation procedures, a rod positioning tool may remain coupled to rod 10 until rod 10 is secured in collars 20 of anchored bone fastener assemblies 15.

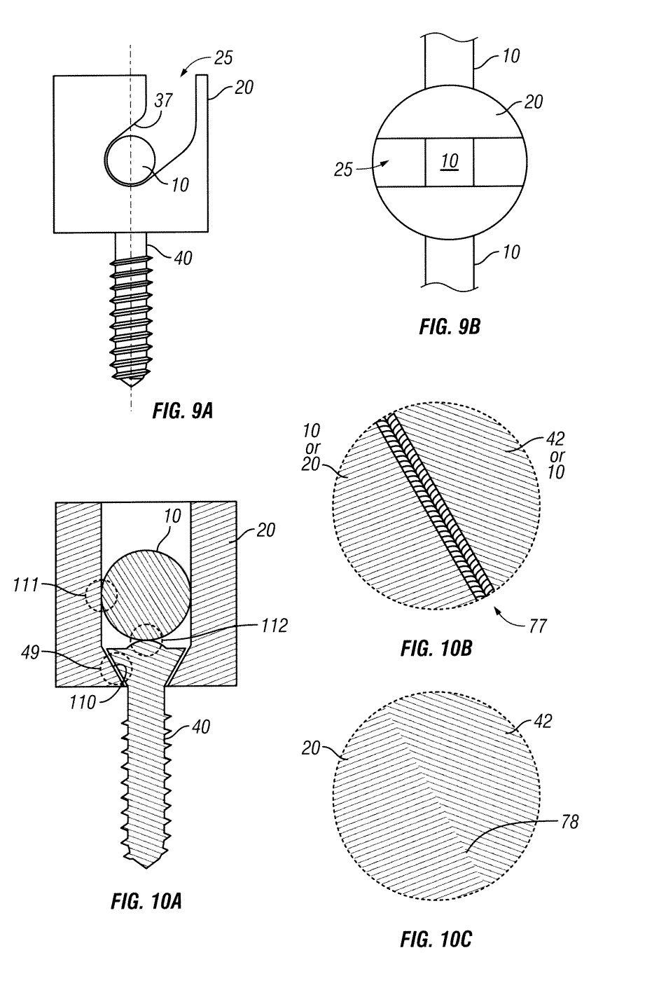

In some embodiments, rod 10 may be advanced into second portion 22 of channel 25. In some embodiments, collar 20 may be rotated about its longitudinal axis to advance rod 10 in second portion 22. FIGS. 9A and 9B depict side and top views of one embodiment of a portion of spine stabilization system 100 in which collar 20 may be rotated to advance rod 10 in collar 20. In some embodiments, collar 20 may be rotated counterclockwise to advance rod 10 in collar 20. In some embodiments, collar 20 may be rotated clockwise to advance rod 10 in collar 20. In some embodiments, a countertorque tool may be used to reduce or eliminate torque applied to the spine during rotation of collars 20. A countertorque tool may attach to rod 10 or bone fastener assembly 15.

In some embodiments, advancing rod 10 in second portion 22 of channel 25 may inhibit withdrawal of rod 10 from collar 20. In some embodiments, advancing rod 10 in collar 20 may cause cold welding between components of spine stabilization system 100. Cold welding may inhibit withdrawal of rod 10 from collar 20. Cold welding may inhibit movement of collar 20 relative to bone fastener 40. Cold welding may inhibit motion of rod 10 relative to bone fastener 40. FIG. 10A depicts a cross-section view of one embodiment of spine stabilization system 100, showing locations 110, 111 and 112 where cold welding may occur when collar 20 is rotated to advance rod 10 in channel 25.

Referring to FIGS. 9A and 10A, cold welding may occur when rod 10 contacts head 42 of bone fastener 40. In some embodiments, as collar 20 is rotated, side 37 of channel 25 may bias rod 10 against head 42. Bone fastener 40 advanced into bone may not move when collar 20 is rotated. In some embodiments, continued rotation of collar 20 may result in an increased force or pressure of surface 36 or 37 of cavity 24 against surface 49 or 48 of bone fastener 40, such as area 110. In some embodiments, zone 77, depicted in FIG. 10B, may form at the interface between collar 20 and head 42 of bone fastener 40. In some embodiments, continued rotation of collar 20 may cause cold welding between collar 20 and head 42 such that collar 20 and head 42 form interface 78, such as depicted in FIG. 10C. In some embodiments, interface 78 may be distinguishable only at the microscopic level. In some embodiments, cold welding between components may result in interface 78 being discontinuous (i.e. the boundary between collar 20 and head 42 may be substantially indistinguishable). In some embodiments, cold welding between collar 20 and bone fastener 40 may inhibit movement of collar relative to bone fastener 40. Inhibiting movement of collar 20 relative to bone fastener 40 may provide rigid support for the spine, may inhibit rotation of collar 20 to inhibit withdrawal of rod 10 from collar 20, and other advantages. In some embodiments, cold welding between collar 20 and bone fastener 40 may occur as a result of the material of collar 20 being softer than the material of bone fastener 40 or the result of material of bone fastener 40 being softer than the material of collar 20. In some embodiments, materials used to manufacture collar 20 or bone fastener 40 may be selected for improved cold welding. For example, commercially pure titanium may be softer than a titanium alloy.

In some embodiments, as collar 20 is rotated, rod 10 may be biased against channel 25 to create area 111 where cold welding may occur. In some embodiments, continued rotation of collar 20 may result in an increased force or pressure of rod 10 against surface 37 of collar 20. In some embodiments, zone 77, depicted in FIG. 10B, may form between collar 20 and rod 10. In some embodiments, continued rotation of collar 20 may cause cold welding between collar 20 and rod 10 such that collar 20 and rod 10 form interface 78, such as depicted in FIG. 10C. In some embodiments, interface 78 may be distinguishable only at the microscopic level. In some embodiments, cold welding between components may result in interface 78 being discontinuous (i.e. the boundary between collar 20 and rod 10 may be substantially indistinguishable). In some embodiments, cold welding between collar 20 and rod 10 may inhibit movement of collar 20 relative to rod 10. Inhibiting movement of collar 20 relative to rod 10 may provide rigid support for the spine, may inhibit translation or rotation of collar 20 relative to rod 10, or other advantages. In some embodiments, cold welding between collar 20 and rod 10 may occur as a result of the material of collar 20 being softer than the material of rod 10 or the result of material of rod 10 being softer than the material of collar 20.

In some embodiments, as collar 20 is rotated, rod 10 may be biased against head 42 of bone fastener 40 to create area 112 where cold welding may occur. In some embodiments, continued rotation of collar 20 may result in an increased force or pressure of rod 10 against head 42 of bone fastener assembly 40. In some embodiments, zone 77, depicted in FIG. 10B, may form between rod 10 and head 42 of bone fastener assembly 40. In some embodiments, continued rotation of collar 20 may cause cold welding between head 42 of bone fastener assembly 40 and rod 10 such that head 42 of bone fastener assembly 40 and rod 10 form interface 78. In some embodiments, interface 78, such as depicted in FIG. 10C, may be distinguishable only at the microscopic level. In some embodiments, cold welding between components may result in interface 78 being discontinuous (i.e. the boundary between head 42 of bone fastener assembly 40 and rod 10 may be substantially indistinguishable). In some embodiments, cold welding between head 42 of bone fastener assembly 40 and rod 10 may inhibit movement of head 42 of bone fastener assembly 40 relative to rod 10. Inhibiting movement of head 42 of bone fastener assembly 40 relative to rod 10 may provide rigid support for the spine, may inhibit translation or rotation of rod 10 relative to head 42 of bone fastener assembly 40, and other advantages. In some embodiments, cold welding between head 42 of bone fastener assembly 40 and rod 10 may occur as a result of the material of head 42 of bone fastener assembly 40 being softer than the material of rod 10 or the result of material of rod 10 being softer than the material of head 42 of bone fastener assembly 40.

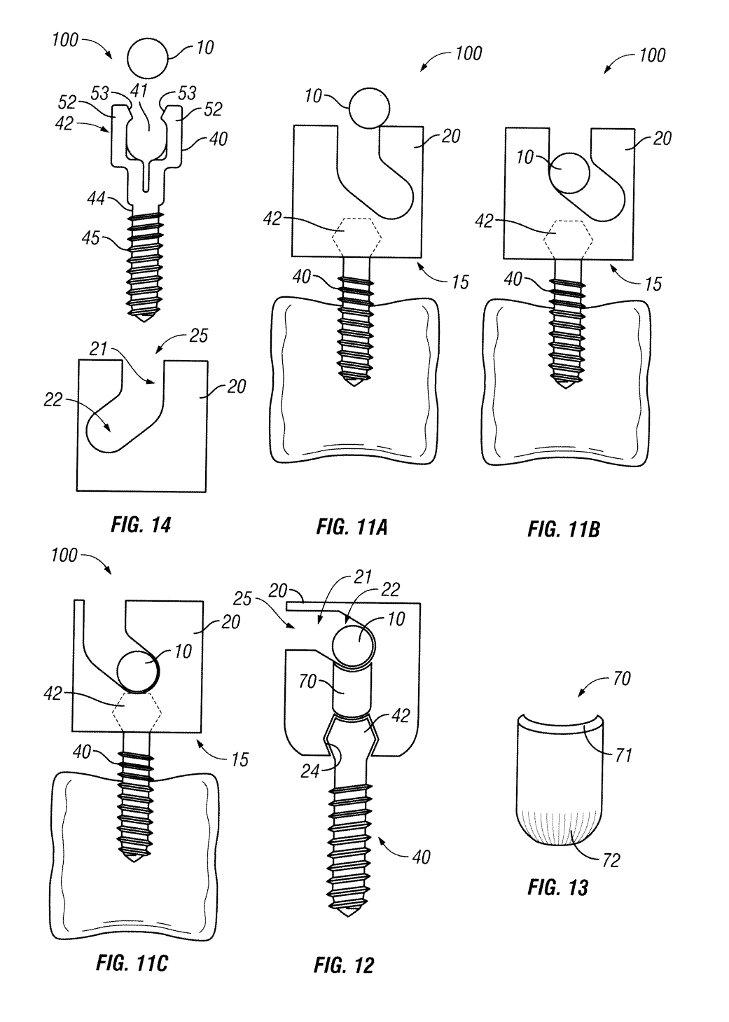

In some embodiments, the angle or orientation of first portion 21 or second portion 22 of channel 25 may inhibit removal of rod 10 from collar 25. As depicted in FIGS. 11A-11C, bone fastener 40 may be advanced into a vertebral body by rotating bone fastener 40 in a first direction. In FIGS. 11A-11C, bone fastener 40 may be considered a left-hand thread such that counterclockwise rotation of bone fastener 40 may engage threads 45 of bone fastener 40 with vertebral bone. Conversely, clockwise rotation of bone fastener 40 may withdraw bone fastener 40 from vertebral bone. FIGS. 11A-11C depict collar 20 having channel 25 oriented opposite bone fastener 40. Thus, rotation of collar 20 in a counterclockwise rotation may enable rod 10 to be inserted or withdrawn from channel 25, and clockwise rotation of collar 20 may cold weld or otherwise secures rod 10 in channel 25. In some embodiments, bone fastener 40 may be advanced into bone by rotating bone fastener 40 in a first direction, and collar 20 may be coupled to bone fastener 40. Rod 10 may be positioned in channel 25. A tool may couple to bone fastener 40. The tool may inhibit rotation of bone fastener 40. Collar 20 may be rotated in a direction opposite the direction in which bone fastener 40 was rotated for advancement into the bone, Rotation of collar 20 about its longitudinal axis may cause cold welding between collar 20 and bone fastener 40. The tool may be uncoupled from bone fastener 40. In these embodiments, any torque exerted on collar 20 by bone fastener 40 may be opposed by a torque exerted on collar 20 by rod 10.

Some embodiments may be useful for coupling rods 10 to bone fasteners 40 in various ways to form spine stabilization system 100 of various heights. FIG. 12 depicts a side view of one embodiment of spine stabilization system 100 in which an intermediary device may be used to facilitate cold welding. In FIG. 12, plunger 70 may be disposed between rod 10 and bone fastener 40. In some embodiments, plunger 70 may be formed from a material that can be cold welded to rod 10 or bone fastener 40 or collar 20. In some embodiments, components of spine stabilization system 100, such as rod 10, collar 20, and bone fastener 40, may be manufactured from material that resists cold welding. In some embodiments, plunger 70 may be disposed between two or more of the components to facillitate cold welding. In some embodiments, plunger 70 may be disposed between components to increase the separation between the components. In some embodiments, plunger 70 may have selected height to provide a desired overall height of bone fastener assembly 15. In FIG. 12, plunger 70 may be disposed between bone fastener 40 and rod 10 to enable rod 10 to be positioned farther away from bone fastener 40 or the vertebra. In some embodiments, plunger 70 may be manufactured from a harder (i.e., less ductile) material than other components of spine stabilization system 100. In some embodiments, plunger 70 may be manufactured such that cold welding may be more likely between one set of components than other components.

FIG. 12 also depicts one embodiment of collar 20 having first portion 21 oriented in a plane perpendicular to the longitudinal axis of collar 40. Instead of positioning rod 10 in channel 25 from a posterior approach, rod 10 may be positioned in channel 25 from a sagittal approach. Rotation of collar 20 about its longitudinal axis may advance rod 10 into second portion 22 of channel 25.

FIG. 13 depicts a perspective view of one embodiment of plunger 70. In some embodiments, plunger 70 may have a first surface 71 for contact with rod 10. In some embodiments plunger 70 may have a second surface 72 for contact with bone fastener 40. In some embodiments, first surface 71 and second surface 72 may be arcuate or angular. In some embodiments, a curvature of first surface 71 may have a different radius than a curvature of second surface 72.

FIG. 14 depicts one embodiment of a portion of spine stabilization system 100. In some embodiments, rod 10 may be advanced into channel 41 formed in head 42 of bone fastener 40. In some embodiments, rod 10 may be positioned between two deflectable opposing arms 52. In some embodiments, head 42 may have a non-circular cross-section. In some embodiments, head 42 may have an oval cross-section. In some embodiments, head 42 may have an elliptical cross-section. In some embodiments, a non-circular cross-section may have a major axis and a minor axis. In some embodiments, cavity 24 formed in collar 20 may have a non-circular profile. In some embodiments, cavity 24 may have an oval cross-section. In some embodiments, cavity 24 may have an elliptical cross-section. In some embodiments, when head 42 is positioned in cavity 24, the major axis of head 42 may be aligned with the major axis of cavity 24 (i.e., and the minor axis of head 42 is aligned with the minor axis of cavity 24) such that opposing arms 52 are in an undeflected state. In some embodiments, rod 10 may be positioned between opposing arms 52 when the major axis is aligned with channel 41. Arms 52 may include protuberances 53 to inhibit withdrawal of rod 10 from channel 41. In some embodiments, positioning of rod 10 in first portion 21 of channel 25 may provisionally lock rod 10 in channel 41. In some embodiments, collar 20 may be rotated to secure rod 10 in channel 41 of head 42 of bone fastener 40. In some embodiments, rotating collar 20 may align the major axis of bone fastener 40 with the minor axis of collar 20 to deflect arms 52 inward. In some embodiments, arms 52 deflected inward may contact rod 10 to inhibit withdrawal of rod 10 from collar 20. In some embodiments, arms 52 deflected inward may contact rod 10 to inhibit motion of rod 10 relative to collar 20. In some embodiments, rotation of collar 20 may advance rod 10 into second portion 22 of channel 25 and may bias arms 52 against rod 10 to inhibit movement of rod 10 relative to collar 20 or head 42 of bone fastener 40.

Minimally invasive procedures may involve locating a surgical site and a position for a single skin incision to access the surgical site. The incision may be located above and between (e.g., centrally between) vertebrae to be stabilized. An opening under the skin may be enlarged to exceed the size of the skin incision. Movement and/or stretching of the incision, bending of a rod, and angulation of collars of bone fastener assemblies may allow the length of the incision and/or the area of a tissue plane to be minimized. In some embodiments, minimally invasive insertion of a spinal stabilization system may not be visualized. In certain embodiments, insertion of a spinal stabilization system may be a top-loading, mini-opening, muscle-splitting, screw fixation technique.

Insertion of a spinal stabilization system may include gradually increasing the diameter of an opening formed in a pedicle and/or vertebral body to accept a bone fastener assembly. For example, a targeting needle may have outer diameter of about D. A bone awl inserted after the targeting needle may have an outer diameter incrementally larger than the outer diameter of the targeting needle. As used herein, an incrementally larger diameter may be large enough to allow a snug but adjustable fit. For example, the bone awl may have outer diameter of about (D+x). A tap portion of a bone tap inserted after the bone awl may have a minor diameter of about (D+2x). A bone fastener may have a minor diameter of about (D+3x). In some embodiments, x may be between about 0.1 mm and about 1.0 mm. For example, x may be about 0.5 mm. Incremental sizing of the targeting needle, bone awl, tap, and bone fastener may promote a proper fit of the bone fastener in the vertebra to be stabilized.

In one embodiment of a spinal stabilization system insertion method, the patient may be placed in a prone position on a radiolucent table with clearance available for a C-arm of a fluoroscope. For example, a Jackson table with a radiolucent Wilson frame attachment may be used. The ability to obtain high quality images is very important. Bolsters, frames, and pads may be inspected for radiolucency prior to the operation. Placing the patient in a knee-chest position (e.g., using an Andrews table) should be avoided. Care should be taken to avoid placing the patient's spine in kyphosis during positioning of the patient.

The C-arm of the fluoroscope should be able to freely rotate between the anteroposterior, lateral, and oblique positions for optimal visualization of pedicle anatomy during the procedure. The arm should be rotated through a full range of motion prior to beginning the procedure to ensure that there is no obstruction or radio-opaque object in the way. The fluoroscope may be positioned so that Ferguson views and “bullseye” views are obtainable. Once the patient is positioned and the ability to obtain fluoroscopic images of the target levels for instrumentation has been confirmed, the patient may be prepared and draped sterilely.

For most of the lumbar region, the vertebral pedicle is an obliquely oriented cylindrical corridor. The angulation varies by approximately 5 degrees per level (e.g., L1: 5 degrees; L5: 25 degrees). A pre-operative fine-cut computed tomography image may be examined to determine any unique anatomy of the patient. Acquiring the pedicle in the most lateral and superior quadrant of the pedicle may be desirable to avoid the overriding facet during a minimally invasive procedure. A lateral entry point may allow for better screw convergence as well as less interference with the superior adjacent level facet joint. A targeting needle may be passed in a medial and inferior trajectory, thus following the natural pathway of the pedicle. Frequent fluoroscopic inspection in both an anteroposterior and lateral plane may ensure proper passage of the needle as the needle is inserted into vertebral bone.

Various techniques may be used to plan the skin incisions and entry points. In one embodiment, the planning sequence for a single-level stabilization may include the following four steps. First, an anteroposterior image may be obtained with the spinous processes centered at the target vertebral bodies. Vertical lines passing through midpoints of pedicles that are to receive bone fasteners may be marked on the patient. The lines do not represent skin entry points. The lines are markers of pedicle entry points used to estimate angles at which targeting needles to be inserted to contact the pedicles. In some embodiments, sets of vertical lines may be drawn corresponding to the lateral edges of the pedicles instead of lines corresponding to the midpoints of the pedicles.

Second, horizontal lines may be marked approximately through the centers of the pedicles (mid-pedicle lines) on the patient. In some embodiments, the lines may be drawn on the superior side of the center axes (superior to the mid-pedicle).

Third, an oblique or “bullseye” view (i.e., down a longitudinal axis of a pedicle) may be obtained on each side of the patient for each pedicle that is to be stabilized. Vertical oblique view lines may be marked on the skin at the midpoints of each of the pedicles that are to receive a bone fastener. The oblique view lines may be drawn in a different color than the vertical lines drawn during the first step. In some embodiments, vertical lines may be drawn corresponding to the lateral edges of the pedicles instead of lines corresponding to the midpoints of the pedicles.

The oblique view lines may be about 2 cm to about 3 cm away from the lateral pedicle border lines marked in the first step. For larger patients, the oblique view line may be greater than about 3 cm away from the midline marked in the first step. For smaller patients, the oblique view line may be closer than about 2 cm away from the midline marked in the first step. The intersection of the oblique view lines with the horizontal lines drawn in the second step may represent skin entry points for a targeting needle as the targeting needle passes through soft tissue at an angle towards the bony pedicle entry point. A side fluoroscopic image, the horizontal lines, and the vertical lines may help the surgeon triangulate between the skin entry points and bony entry points.

Fourth, an incision may be made in the skin between mid-pedicle lines along the vertical oblique view lines. The skin incision may be from about 2 cm to about 4 cm long. In some embodiments, the incision may be from about 2.5 cm to about 3 cm long. Limiting the length of the incision may enhance patient satisfaction with the procedure. The incisions may be pre-anesthetized with, for example, 1% lidocaine with 1:200,000 epinephrine. To blunt the pain response, a long spinal needle may be used to dock on the bone entry point and inject the planned muscle path in a retrograde fashion as well. Once the incision has been made, tissue surrounding the incision may be pulled and/or stretched to allow access to a target location in a vertebra.

After sterile preparation and draping, the pedicle entry points may be fluoroscopically rechecked to ensure that the previously marked lines correspond to the intersection of the midline of the transverse process and the lateral joint and pars interarticularis. The intersection of the facet and the transverse process provides a starting point that may help avoid the canal and follow the natural inclination of lumbar pedicles. For the spinal stabilization system described, in which sleeves coupled to bone fastener assemblies are substantially unconstrained by insertion angles of the bone fasteners, patient anatomy may determine the most advantageous insertion angles of the bone fasteners.

A scalpel may be used to make a stab wound at the junction of an oblique view line and a mid-pedicle line. In one embodiment, the scalpel may be a #11 scalpel. A targeting needle may be passed through the incision in an oblique lateral to medial trajectory towards the bony entry point defined by a lateral pedicle border line. The C-arm of the fluoroscope may be placed in an anteroposterior position for this maneuver.

As the targeting needle encounters the bony anatomy, anteroposterior fluoroscopic images may be used to place the tip of the needle at the upper outer quadrant of the pedicle. In some embodiments, the needle may be walked medially along the transverse process to the pedicle entry point. In some embodiments, the needle tip may be docked by lightly tapping the tip into the bone with a mallet or other impact device to drive the tip into the bone. In some embodiments, the needle tip may be docked by applying downward pressure to the targeting needle to force the tip into the bone.

The fluoroscope may then be moved to a lateral position. The surgeon may correct the sagittal trajectory of the needle by moving the needle in an anterior or posterior direction to match the vector of the pedicle corridor. In some embodiments, a mallet or other impact device may be used to gently advance the targeting needle into the pedicle halfway to the pedicle-vertebral body junction. In other embodiments, force may be applied to the targeting needle to drive the targeting needle into the pedicle halfway to the pedicle-vertebral body junction. An anteroposterior image may then be obtained to confirm that the needle is approximately halfway across the pedicle in the anteroposterior view. If the tip is more than halfway across the pedicle in a lateral to medial projection, the trajectory may be too medial. Further advancement of the needle may risk passing the needle through the spinal canal. The needle may be repositioned. A new starting point or new trajectory may be obtained. If the anteroposterior image demonstrates that the needle is significantly lateral in the pedicle, then the needle may have passed along the lateral portion of the pedicle. A needle that has passed along the lateral portion of the pedicle may be withdrawn and repositioned.

Once a good trajectory has been obtained, the targeting needle may be advanced using a mallet. In some embodiments, the needle may be pushed in without a mallet. The targeting needle may be advanced to the junction of the pedicle and vertebral body under lateral fluoroscopic guidance.

A scale on a targeting needle may be used to approximate a length of a bone fastener to be used. A first depth of a targeting needle may be measured relative to the body surface when a pedicle is first encountered. A second depth of the targeting needle may be measured relative to the body surface after the targeting needle has been advanced to the desired depth in a vertebral body. An approximate length of the pedicle screw to be used may be determined by taking a difference between the depth measurements.

Once the guide wire has been passed through the targeting needle and the targeting needle has been removed, the guide wire may be used as a guide to position one or more successively sized dilators around a target location in a pedicle. A dilator may be a conduit with a regular shape (e.g., cylindrical) or an irregular shape (e.g., C-shaped). A dilator may form an opening through soft tissue to the pedicle. For patients with a thick fascia, it may be advantageous to make a nick in the fascia with a scalpel blade to facilitate passage of the dilators. The dilators may be passed sequentially over the guide wire. The dilators may be rotated during insertion to facilitate dilation of surrounding tissue. The dilators may be inserted until the leading edges contact the pedicle. A distal end of a dilator may be tapered to facilitate positioning of the dilator proximate the pedicle. An instrumentation set for a spinal stabilization system may include two, three, four, or more successively sized dilators.

In some embodiments, a first dilator may have an inner diameter just slightly larger than an outer diameter of a guide wire. As used herein, “an inner diameter just slightly larger than an outer diameter” may mean that the inner diameter is between about 0.03 mm and about 1.0 mm greater than the outer diameter. For example, an inner diameter of a first dilator may be about 0.5 mm greater than the outer diameter of a guide wire. Lengths of dilators in a successively sized set may decrease with increasing diameter to facilitate removal of the smaller dilators. Care should be taken to avoid dislodging a guide wire during insertion and removal of the dilators.

After tissue dilation has been achieved, a large diameter dilator may be used to guide a bone fastener assembly and/or insertion instruments toward a target location in a pedicle. The bone fastener and/or insertion instruments may be advanced through the tissue dilator to the vertebrae to be stabilized. In some embodiments, an initial passage may be formed in the pedicle and the vertebral body using a drill or a drill and tap combination.

In some embodiments, a length of the threaded portion of a tap may be used to determine a depth of a threaded passage formed in a bone. For a threaded portion of a known length (e.g., 30 mm, 45 mm, 60 mm), a scaled image (e.g., X-ray image) of a depth of the threaded portion in a bone monitored during tapping may allow a medical practitioner to determine the depth of the threaded passage. In some embodiments, a tap may form threads of major diameter about 0.5 mm smaller than a major diameter of threads of a bone fastener to be inserted into the threaded passage.

After a threaded passage of a desired length has been formed in a vertebral body, a second measurement of the position of the tap relative to a top of a dilator may be determined using indicia on the tap. A length of a threaded member may be determined by taking a difference between the first and second measurements. In some embodiments, an estimate of length may be derived based upon fluoroscopic images and a known length of the tap that is visibly recognizable in the fluoroscopic images. The tap may be removed from vertebral body and pedicle by rotating the tap out of the vertebral body and the pedicle.

A bone fastener assembly with a bone fastener of an appropriate length may be selected for insertion in a patient. The size of the bone fastener may be verified with measurement indicia in an instrumentation set. In some embodiments, measurement indicia may be etched or printed on a portion of an instrumentation set. For example, the chosen bone fastener embodiment may be placed over the outline of a bone fastener embodiment printed on a tray of the instrumentation set.

The chosen bone fastener assembly may be attached to a detachable member. In one embodiment, a bone fastener assembly may be rotated on a flange of a detachable member. Movable members of the detachable member may be extended into indentations in a collar of the bone fastener assembly. A driver may be used to extend the movable members to couple with the collar. When the bone fastener assembly is coupled to the detachable member, a drive portion of a fastener driver may be coupled to a tool portion of the bone fastener. A shaft of the fastener driver may be positioned in the passage of the detachable member. A removable handle may be attached to the shaft of the fastener driver. The detachable member, collar, and bone fastener may be substantially co-axial when the fastener driver is positioned in the detachable member. In some embodiments, the removable handle may be attached to the shaft of the fastener driver after the bone fastener, collar, detachable member, and fastener driver combination is positioned down a guide wire through a dilator and against a pedicle.

In some embodiments, a driver coupled to bone fastener 40 and a sleeve may be inserted along a guide wire into a dilator. The guide wire may represent the trajectory that bone fastener 40 or bone fastener assembly 15 may follow toward a pedicle during insertion of a spinal stabilization system. In some embodiments, tissue surrounding the incision may be pulled and/or stretched to allow a desired angular orientation of the bone fastener assembly relative to a pedicle. After insertion of bone fastener assembly 15, the sleeve, and the driver in the dilator, the driver may be rotated to thread bone fastener 40 into the pedicle and vertebral body. The bone fastener may be advanced into the pedicle under fluoroscopic guidance to inhibit breaching of the pedicle walls. When the tip of the bone fastener advances beyond the posterior margin of the vertebral body, the guide wire may be removed to inhibit inadvertent bending of the guide wire or unwanted advancement of the guide wire.

In some embodiments, bone fastener 40 may be advanced to bring collar 20 down snug to the facet joint. In some embodiments, bone fastener 40 may then be backed off about a quarter of a turn. Backing bone fastener 40 off about a quarter of a turn may allow for full motion of collar 20 relative to bone fastener 40. After bone fastener 40 has been advanced to the desired depth, the driver may be removed from head 42 of bone fastener 40 and from the dilator. After removal of the driver, the dilator may be removed from the patient.

After bone fastener 40 has been secured to the vertebra and the driver has been removed from the sleeve, collar 20 may allow angulation of the sleeve relative to bone fastener 40. Tissue surrounding the incision may be released such that the sleeve is angled toward a central location between vertebrae to be stabilized. The sleeve may be moved to facilitate positioning of instruments and/or to facilitate access to the adjacent vertebra that is to be stabilized. For example, the sleeve may be tilted towards the adjacent pedicle so that additional length of an opening in the patient is not needed. The channel in the sleeve may be turned toward the adjacent pedicle that is to be stabilized with the spinal stabilization system being formed.

A plane of dilated tissue may be created between a first pedicle and a second pedicle to be stabilized with a spinal stabilization system. In some embodiments, bone fastener assembly 15 and a sleeve may be coupled to the first pedicle. The second pedicle may be adjacent to the first pedicle. In one embodiment, a tissue wedge may be placed in the sleeve coupled to the first pedicle such that the distal end of the tissue wedge contacts the head 42 of bone fastener 40. The proximal end of the sleeve coupled to the first pedicle may be held such that tissue around the incision is not pulled or stretched. The tissue wedge may be wanded through the channel in the sleeve and channel 25 in collar 20 toward the target location at the second pedicle, thereby creating a plane in muscle and other tissue between head 42 of installed bone fastener 40 and the target location of a second bone fastener 40. In some embodiments, a tissue wedge may be pivoted about an inside proximal edge of the sleeve such that the distal end of the tissue wedge bluntly splits the muscle and fascia along fibers and create a tissue plane between the two pedicles. The wanding action may be repeated more than once (e.g., two or three times) to create a good working plane and displace unwanted tissue from the plane. The wanding may create a tissue plane. In some embodiments, the tissue plane may be substantially trapezoidal. In certain embodiments, a tissue plane may be created before bone fastener assembly 15 is inserted into a vertebra.

A tissue plane may be made in a variety of shapes including, but not limited to, substantially trapezoidal, substantially rhomboidal, and substantially triangular. A tissue plane with a substantially geometric shape may have the basic geometric shape with, for example, slightly curved edges and/or slightly rounded corners or apices. In some embodiments, a sleeve length may be chosen to reduce a size of a tissue plane that needs to be formed between pedicles. In certain embodiments, creating a trapezoidal tissue plane may reduce the invasiveness of a procedure. Limiting the area of the plane may promote a faster recovery time and/or may reduce an amount of post-operative pain experienced by the patient.

In one embodiment, a tissue wedge may be coupled to a portion of a sleeve to facilitate creation of a tissue plane. In one embodiment, two pedicles may be targeted and bone fastener assemblies anchored in both pedicles before creation of a tissue plane. A tissue wedge may be inserted at either of the pedicles. In some embodiments, the sleeves may be coupled to each other at proximal ends of the sleeves. The tissue wedge may be coupled to a sleeve and the sleeve may be used as an anchor during wanding. Insertion of rod 10 into collars 20 of bone fastener assemblies 15, however, may require cutting of some tissue between the two sleeves.

Other procedures may be used to create a tissue plane. For example, before targeting pedicle locations, a tissue wedge may be worked downward from an incision to create a tissue plane. Alternatively, a scalpel may be used to cut from the surface of the body to vertebral bone. Extensive use of a scalpel, however, may remove benefits of a minimally invasive procedure.

In one embodiment, a targeting needle may be passed through the tissue to create a tissue plane for insertion of a rod. The shaft of the targeting needle may be wanded from a sleeve in a first pedicle to a target location in a second pedicle to separate the soft tissue in a plane between the pedicles. After the targeting needle contacts the second pedicle and the plane is established, bone fastener assembly 15 may be inserted in the second pedicle using a procedure similar to the procedure used to place a second bone fastener assembly 15 in an adjacent pedicle.

Once a well-defined tissue plane has been formed, a targeting needle may be passed down a first sleeve coupled to a first vertebra and then wanded along the formed plane over to a target location at a second pedicle. The target location at the second pedicle may be fluoroscopically confirmed. A bone fastener assembly coupled to a sleeve may be secured in the second pedicle using a procedure similar to the procedure used to insert a bone fastener assembly in a first pedicle.

With bone fastener assemblies 15 secured in the vertebral bodies, sleeves coupled to bone fastener assemblies 15 may be oriented to facilitate insertion of rod 10 in the sleeves. In some embodiments, sleeves may serve as tissue retractors during a spinal stabilization procedure. Angular motion of collar 20 may be limited by a range of motion allowed between collar 20 and bone fastener 40 that collar 40 is anchored to. Angular motion of collar 20 may be limited by patient anatomy. Angular motion or orientation of one collar 20, however, may not depend upon a position of another collar 20. In some embodiments, channel openings in the sleeves may face each other. In other embodiments, channel openings in the sleeves may be angled relative to each other in various arrangements. A distance between the sleeves may be estimated using an estimating tool. The distance between the sleeves may be used to select a length of rod 10 needed to couple collars 20.

In one embodiment, flexible arms of an estimating tool may be positioned in sleeves. With the activator disengaged, the estimating tool may be advanced toward the pedicles until the arms or members rest on the collars or bone fasteners of the bone fastener assemblies. The activator may be engaged. When the arms are withdrawn from the sleeves, a biasing element may allow the arms to extend to the length indicative of the distance between bone fastener assemblies. A rod length may be selected by measuring a distance between the members of the estimating tool. The measured distance may be increased by an amount to allow the rod to extend beyond the collars after curvature and/or insertion. In one embodiment, about 5 mm to about 30 mm (e.g., about 15 mm) may be added to the measured distance. In some embodiments, a desired length of a rod may be a length that allows the rod to extend from each collar by about 2 mm or about 3 mm. In certain embodiments, ends of a rod may be flush with the outer surface of one or more collars.

In one embodiment, rod 10 of desired length may be chosen by estimating a distance between the sleeves without the use of an estimating tool. The sleeves may be positioned as desired (e.g., substantially parallel to each other). A distance between the most distant outer edges of the sleeves may be estimated. The estimated distance may be increased by an amount to allow rod 10 to extend beyond collars 20 after insertion. In some embodiments, from about 1 mm to about 20 mm may be added to the estimated distance. In some embodiments, a desired length of rod 10 may be a length that allows rod 10 to extend from each collar 20 by about 2 mm.

Rod 10 may be cut to length and contoured as desired. For example, a medical practitioner may use experience and judgment to determine curvature of rod 10 for a patient. A desired curvature for rod 10 may be determined using fluoroscopic imaging. In some embodiments, a curvature of rod 10 may be chosen such that, when rod 10 is secured to collars 20 of bone fastener assemblies 15, sleeves coupled to bone fastener assemblies 15 cross at a surface of the skin. Crossing of the sleeves at a surface of the skin allows the medical practitioner to minimize trauma to a patient by minimizing incision length and tissue plane area. In some embodiments rod 10 may be bent or shaped with a tool (e.g., a rod bender) to allow insertion of rod 10 through channels of sleeves with various spatial locations and/or various angular orientations.

Rods 10 may have shapes including, but not limited to, straight, bent, curved, s-shaped, and z-shaped. In some embodiments, rods 10 may have a substantially circular longitudinal cross section. In certain embodiments, rods 10 may have other cross-sectional shapes including, but not limited to, regular shapes (oval, rectangular, rhomboidal, square) and irregular shapes. An instrumentation kit for a spinal stabilization system may include straight rods and/or pre-shaped rods. Straight rods and/or pre-shaped rods may be contoured to accommodate patient anatomy if needed during the surgical procedure.

Channels of the sleeves and channels 25 of collars 20 may be oriented by rotating the sleeves to accommodate insertion and advancing of rod 10. In certain embodiments, a channel opening in a sleeve may be non-linear (e.g., bent, curved, or angled) to allow portions of the spine to be selectively stabilized. Sleeve orientation and/or design may be chosen to allow compression, distraction, and/or reduction of vertebrae. In some embodiments, there may be no constraints governing relative location and/or orientation of the sleeves. Sleeves may be forced apart or angled toward each other or away from each other to accommodate insertion of rod 10.

Prior to insertion of rod 10, the tissue wedge or targeting needle may be used to wand between bone fasteners 40 to ensure a clean plane between bone fastener assemblies 15. An end of rod 10 may be inserted at an angle or substantially longitudinally in a passage and/or channel of a sleeve coupled to bone fastener assembly 15. Inserting rod 10 at an angle or substantially longitudinally allows the length of the incision and/or the area of the tissue plane to remain advantageously small. In some embodiments, sleeves coupled to anchored bone fastener assemblies 15 may remain essentially unconstrained relative to each other during insertion of rod 10. In certain embodiments, angular orientation of collars 20 may determine a trajectory of rod 10 down the sleeves and into collars 20 of bone fastener assemblies 15. Inserting rod 10 down two or more sleeves and through an open path (i.e., the tissue plane) may allow a medical practitioner to avoid surgical difficulties associated with anatomical abnormalities and/or misalignment of system components (e.g., in multi-level stabilization procedures).

Insertion of rod 10 may not be visualized subcutaneously. Therefore, a positioning tool may be used to guide rod 10 down the sleeves into channels 25 in collars 20. A distal portion of the positioning tool may be contoured. The contour may allow for some rotation of rod 10. With slight pressure, rod 10 may be rotated subcutaneously into a substantially horizontal position and positioned in first portion 21 of channels 25. The positioning tool may be held firmly while still allowing a rocking movement between rod 10 and the distal end of the positioning tool. Movement of rod 10 may allow rod 10 to be maneuvered down the sleeves and into first portion 21 of channels 25 in collars 20.

In some embodiments, channels 25 in collars 20 may be aligned with channels in the sleeves to allow rod 10 to be positioned in collars 20. A positioning tool may be used to translate rod 10 through channel 25 such that an end of the rod protrudes through collar 20. With one end of rod 10 extending through channel 25 in collar 20, the positioning tool may be used to guide the other end of rod 10 the remaining distance down a second sleeve. The positioning tool may then be used to position the second end of rod 10 in first portion 21 of channel 25. The distal end of a positioning tool may be contoured (e.g., curved and/or grooved) to allow some motion (e.g., rocking) of rod 10 while rod 10 is coaxed into position and/or rotated subcutaneously with the positioning tool. Pressure may be applied to position rod 10 in first portions 21 of channels 25.

After a rod has been positioned in first portion 21 of channel 25 and advanced into second portion as desired, collars 20 may be rotated to couple rod 10 to collars 20. One or more torque wrenches may be attached to collars 20. Collars 20 may be rotated a desired angle. A torque wrench may be oriented at a first position to indicate when channel 25 is aligned with rod 10 (i.e. rod 10 may be inserted into or withdrawn from collar 20). A torque wrench may be oriented at a second position to indicate when channel 25 is at some angle with rod 10 (i.e. rod 10 is securely coupled to collar 20). A torque wrench may include indicia or a scale regarding the amount of torque applied to collar 20. A surgeon may use the information provided by the indicia or scale to determine whether components in spine stabilization system 100 experience cold welding. Torque applied to collar 20 may bias rod 10 against head 42 of bone fastener 40. Torque applied to collar 20 may bias first side 37 of channel 25 against rod 10. The combination of collar biasing rod 10 against head 42 of bone fastener 40 and biasing first side 37 of channel 25 against rod 10 may bias surfaces 36 or 39 of collar 20 against surface 48 or 49 of bone fastener 40. Continued rotation of collar 20 may cause cold welding to occur between head 42 and collar 20.

Torque required to rotate collar 20 may be a source of pain and/or injury to a patient. In some embodiments, a rod 10 may be held with a counter torque wrench as collar 20 is rotated. A counter torque wrench may inhibit or reduce transfer of torque to the patient's spine.

In some embodiments, a spinal stabilization system may be inserted using an invasive procedure. Since insertion of a spinal stabilization system in an invasive procedure may be visualized, cannulated components (e.g., bone fasteners) and/or instruments (e.g., detachable members) may not be needed for the invasive (i.e., open) procedure. Thus, a bone fastener used in an invasive procedure may differ from a bone fastener used in a minimally invasive procedure.

In some embodiments, tools used in an invasive procedure may be similar to tools used in a minimally invasive procedure. In certain embodiments, methods of installing a spinal stabilization system in an invasive procedure may be similar to methods of installing a spinal stabilization system in a minimally invasive procedure.

Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the disclosure. It is to be understood that the forms of the disclosure shown and described herein are to be taken as the presently preferred embodiments. Elements and materials may be substituted for those illustrated and described herein, parts and processes may be reversed, and certain features of the disclosure may be utilized independently, all as would be apparent to one skilled in the art after having the benefit of this description of the disclosure. Changes may be made in the elements described herein without departing from the spirit and scope of the disclosure as described in the following claims.

What is claimed is:

1. A collar for coupling a spinal rod to a bone fastener, comprising:
a first end comprising:
an opening for accommodating a shank of a bone fastener; and
a cavity recessing from the opening into the first end for accommodating a head of the bone fastener;
a second end having a channel formed therein, wherein the channel comprises:
a first portion having a first interior geometric configuration to allow a rod to move relative to the collar, wherein the first portion is oriented in a plane at an angle relative to the longitudinal axis of the collar; and
a second portion oriented relative to the longitudinal axis of the collar,
wherein the second portion has a second interior geometric configuration having a first side for biasing the rod against the head of a bone fastener and a second side for biasing the collar against the head of the bone fastener to cause cold welding of a surface of the cavity of the collar and a surface of the bone fastener and inhibit movement of the collar relative to the bone fastener.

2. The collar of claim 1, wherein the biasing of the first side of the collar and the surface of the rod further inhibits movement of the collar relative to the rod.

3. The collar of claim 1, wherein the first interior geometric configuration of the first portion of the channel comprises a width to provisionally lock the rod in the collar.

4. The collar of claim 3, wherein the first portion of the channel is oriented parallel with the longitudinal axis of the collar.

5. The collar of claim 3, wherein the first portion of the channel is oriented perpendicular to the longitudinal axis of the collar.

6. The collar of claim 1, wherein the second geometric configuration of the second portion of the channel comprises a circular helix oriented about the longitudinal axis of the collar, wherein the bias applied to the rod is proportional to a pitch and an arclength of the circular helix.

7. The collar of claim 1, wherein the second portion of the channel is oriented perpendicular to the longitudinal axis of the collar, wherein the bias applied to the rod is based on the width of the channel.

8. The collar of claim 7, wherein the second portion of the channel has a first width, wherein the channel further comprises a recessed portion having a second width, wherein the second width is wider than the first width to inhibit removal of the rod.

9. The collar of claim 7, wherein the second portion of the channel has a first width, wherein the channel further comprises a protuberance having a second width, wherein the second width is narrower than the first width to inhibit removal of the rod.

10. A method for coupling a rod to a bone fastener having a head and a shank, comprising:
advancing the bone fastener into a first end of a collar, wherein the first end of the collar comprises:
an opening for accommodating the shank of the bone fastener; and
a cavity recessing from the opening into the first end for accommodating the head of the bone fastener;
positioning a rod in a first portion of a channel in a second end of the collar, wherein the first portion has a first interior geometric configuration to allow the rod to move relative to the collar, wherein the first portion is oriented in a plane at an angle relative to the longitudinal axis of the collar;
advancing the rod into a second portion of the channel, wherein the second portion is oriented relative to the longitudinal axis of the collar and wherein the second portion has a second interior geometric configuration having a first side for biasing the rod against the head of a bone fastener and a second side for biasing a surface of the cavity of the collar against the head of the bone fastener, and
rotating the collar a selected angle about its longitudinal axis to bias the surface of the head of the bone fastener against the surface of the cavity, causing cold welding between a portion of the surface of the bone fastener and a portion of the surface of the cavity to inhibit movement of the collar relative to the bone fastener.

11. The method of claim 10, wherein rotating the collar a selected angle about its longitudinal axis biases a surface of the rod against a surface of the channel, causing cold welding between a portion of the surface of the rod and a portion of the surface of the collar to inhibit movement of the collar relative to the rod.

12. The method of claim 10, wherein advancing the rod in the first portion of the channel provisionally secures the rod in the collar.

13. The method of claim 10, wherein the second geometric configuration comprises a circular helix oriented about the longitudinal axis of the collar, wherein rotating the collar applies the bias to the rod proportional to a pitch and an arclength of the circular helix.

14. The method of claim 10, wherein the second portion is oriented perpendicular to the longitudinal axis of the collar, wherein rotating the collar applies an axial bias to the rod corresponding to the width of the channel.

15. The method of claim 10, wherein the steps are part of a minimally invasive surgery (MIS) procedure.

16. A system for stabilizing a portion of a spine, comprising:
a rod having a length for spanning between two or more vertebrae;
a plurality of bone fasteners, wherein each bone fastener comprises:
a head; and
a threaded shank for advancement into one of the two or more vertebrae; and
a collar comprising:
a first end comprising:
an opening for accommodating the shank of the bone fastener; and
a cavity recessing from the opening into the first end for accommodating the head of the bone fastener;
a second end having a channel formed therein, wherein the channel comprises:
a first portion having a first interior geometric configuration to allow a rod to move relative to the collar,
wherein the first portion is oriented in a plane at an angle relative to the longitudinal axis of the collar; and
a second portion oriented relative to the longitudinal axis of the collar,
wherein the second portion has a second interior geometric configuration having a first side for biasing the rod against the head of a bone fastener and a second side for biasing the collar against the head of the bone fastener to cause cold welding of a surface of the cavity of the collar and a surface of the bone fastener and inhibit movement of the collar relative to the bone fastener.

17. The system of claim 16, wherein the bone fastener comprises:
a head having an elliptical cross-section with a major axis and a minor axis and comprising:
a channel aligned with the minor axis and formed by two opposing arms along the major axis; and
a slot;
wherein the cavity in the collar has an elliptical cross-section having a major axis and a minor axis,
wherein the width of the cavity on the major axis of the cavity is greater than the width of the head on the major axis of the head and the width of the cavity on the minor axis of the cavity is substantially equal to the width of the head on the major axis of the head,
wherein a rod is securely coupled to the bone fastener and collar when the minor axis of the cavity is substantially aligned with the major axis of the head.

18. The system of claim 16, wherein the biasing of the first side of the collar and the surface of the rod further inhibits movement of the collar relative to the rod.

19. The system of claim 16, wherein the second geometric configuration of the second portion of the channel comprises a circular helix oriented about the longitudinal axis of the collar, wherein the bias applied to the rod is proportional to a pitch and an arclength of the circular helix.

20. The system of claim 16, wherein the second portion of the channel is oriented perpendicular to the longitudinal axis of the collar, wherein the bias applied to the rod is based on the width of the channel.
